(19) United States
(12) Patent Application Publication
(10) Pub. No.: US 2019/0309272 A1
(43) Pub. Date: Oct. 10, 2019

(54) Genetically Modified Cell Lines Including a TP53 Modification and Methods of Use

(71) Applicant: UNIVERSITY OF SOUTH CAROLINA, COLUMBIA, SC (US)

(72) Inventors: CAROLYN E. BANISTER, COLUMBIA, SC (US); JOHN WARRINGTON, MOORESTOWN, NJ (US); CHANGLONG LIU, COLUMBIA, SC (US); PHILLIP BUCKHAULTS, COLUMBIA, SC (US)

(21) Appl. No.: 16/377,960

(22) Filed: Apr. 8, 2019

Related U.S. Application Data
(60) Provisional application No. 62/654,799, filed on Apr. 9, 2018.

(51) Int. Cl. C12N 9/12; C12Q 1/6886; C12N 15/10

(52) U.S. Cl. CPC C12N 9/1205; C12Q 1/6886; C12N 15/1024; C12Q 2600/156; C12N 2310/20; C12N 2800/80; C12Q 2600/106; C12Y 207/01037

(57) ABSTRACT

The present disclosure is directed to genetically engineered cell lines which include a modification to knockout a portion of the TP53 gene. Embodiments disclosed herein provide aspects of the knockout cell lines, methods for producing the knockout cell lines, in vitro assays using the knockout cell lines, and kits including the knockout cell lines. In certain implementations, the embodiments can provide doctors and patients improved tools for determining a treatment or for comparing treatments for patients having tumors that include a TP53 mutation.

### CROSS REFERENCE TO RELATED APPLICATION

This application claims filing benefit of U.S. Provisional Application Ser. No. 62/654,799, having a filing date of Apr. 9, 2018, which is incorporated herein by reference in its entirety.

### STATEMENT AS TO RIGHTS TO INVENTIONS MADE UNDER FEDERALLY SPONSORED RESEARCH

This invention was made with Government support under Contract No. U01 CA158428, awarded by the National Institutes of Health (NIH). The Government has certain rights in the invention.

### BACKGROUND

Breast cancer is the most common cancer diagnosed among women and the second leading cause of cancer death for women in the United States. Due to genetic variation in cancer cells, about 36.1% of breast cancers acquire a loss of function mutation to the tumor suppressor gene, TP53, yet few therapies have been developed for targeting the absence of TP53.

Assaying new therapies commonly uses a cell line, such as the MCF7 breast cancer cell line, to determine drug efficacy in vitro before moving to animal or human studies. Though advancements in genetic editing such as CRISPR-Cas9 can allow for accurate and precise engineering of genomic DNA, no known studies have focused on genetically modifying a cancer cell line to modify the TP53 gene for use in assaying new therapies.

### SUMMARY

The present disclosure is directed to genetically engineered cell lines which include a modification to knockout a portion of the TP53 gene. Embodiments disclosed herein provide aspects of the knockout cell line, methods for producing the knockout cell line, in vitro assays using the knockout cell line, and kits including the knockout cell line. In certain implementations, the embodiments can provide doctors and patients improved tools for determining a treatment or for comparing treatments for patients having tumors that include a TP53 mutation.

An example embodiment of the disclosure includes a knockout cell line composed of MCF7 breast cancer cells having decreased endogenous expression of at least one coding region in the tumor protein 53 (TP53) gene having a nucleotide sequence corresponding to Seq. ID No. 1. Generally, the cells of the knockout cell line include a genetic modification to remove or delete a portion of the TP53 gene which results in decreased endogenous expression of the at least one coding region.

In certain embodiments, the portion of the TP53 gene can includes one or more exons that encode a portion of the messenger RNA (mRNA) for producing the TP53 protein. Each of these exons includes a nucleotide sequence corresponding to continuous sequence of base pairs from Seq. ID No. 1 as shown in Table 2. Thus, the coding regions can include one or more of exons 1-11.

In some embodiments, the coding region of the TP53 gene can include part of one exon. For example, an embodiment of the disclosure can include a knockout cell line where each cell includes a modification to delete part of one exon from Seq. ID No. 1. As an example implementation, a knockout cell line of the disclosure can include removing at least part of exon 4 including the sequence: GGACGATATT GAACAATGGT TCACTGAAGA CCCAGGTCCA GATGAAGCTC CCAGAATGCC AGAGGCTGCT CCCCCCGTGG CCCCTGCACC AGCAGCTCCT ACACCGGCGG CCCCTGCACC AGCCCCCTCC TGGCCCCTGT CATCTTCTGT CCCTTCCCAG AAAACCTACC AGGGCAGCTA CGGTTTCC.

As used herein, each of exons on 1-11 include a nucleotide sequence from Seq. ID No. 1 that corresponds to a range of base numbers for each exon. For example, the nucleotide sequence for exon 1 comprises base numbers 1-162; the nucleotide sequence for exon 2 comprises base numbers 10917-11018; the nucleotide sequence for exon 3 comprises base numbers 11136-11157; the nucleotide sequence for exon 4 comprises base numbers 11267-11545; the nucleotide sequence for exon 5 comprises base numbers 12303-12486; the nucleotide sequence for exon 6 comprises base numbers 12568-12680; the nucleotide sequence for exon 7 comprises base numbers 13249-13358; the nucleotide sequence for exon 8 comprises base numbers 13702-13838; the nucleotide sequence for exon 9 comprises base numbers 13931-14004; the nucleotide sequence for exon 10 comprises base numbers 16824-16930; and the nucleotide sequence for exon 11 comprises base numbers 17849-19137.

An example embodiment of the disclosure can include a knockout cell line of MCF7 breast cancer cells that include a deletion of the nucleotide sequence for exon 4 from Seq. ID No. 1, the nucleotide sequence for exon 4 including: TCCC CCTTGCCGTC CCAAGCAATG GATGATTTGA TGCTGTCCCC GGACGATATT GAACAATGGT TCACTGAAGA CCCAGGTCCA GATGAAGCTC CCAGAATGCC AGAGGCTGCT CCCCCCGTGG CCCCTGCACC AGCAGCTCCT ACACCGGCGG CCCCTGCACC AGCCCCCTCC TGGCCCCTGT CATCTTCTGT CCCTTCCCAG AAAACCTACC AGGGCAGCTA CGGTTTCCGT CTGGGCTTCT TGCATTCTGG GACAGCCAAG TCTGTGACTT GCACG.

Another example embodiment of the disclosure includes a knockout cell line of MCF7 breast cancer cells that include a deletion of the nucleotide sequence for exon 10, the nucleotide sequence for exon 10 including: ATCCGTG GGCGTGAGCG CTTCGAGATG TTCCGAGAGC TGAATGAGGC CTTGGAACTC AAGGATGCCC AGGCTGGGAA GGAGCCAGGG GGGAGCAGGG CTCACTCCAG.

An embodiment of the disclosure can also include an in vitro assay for determining the efficacy of a treatment in breast cancer cells that include a TP53 gene mutation. In an example implementation, the method can include: providing the treatment to a group of cells derived from a knockout cell as exemplified in certain embodiments of the disclosure and measuring a result. In certain implementations of the invitro assay, measuring the result can include determining a quantitative measure of cell death. Alternatively or additionally, in some implementations providing the treatment can include administering a drug to the plurality of cells derived from the knockout cell line. Non-limiting examples of the drug can include one or more of the compounds listed in Table 2. Further, in certain implementations administering the drug can include administering: Nutlin3, Fluorouracil, Palbociclib, or combinations thereof.

In an example embodiment of the in vitro assay, determining the efficacy of the treatment can also include providing the treatment to a group of wild type MCF7 breast cancer cells and comparing the treatment between the wild type MCF7 breast cancer cells and the cells derived from the knock-out cell line. As an example implementation, comparing the treatment between the wild type MCF7 breast cancer cells and the cells derived from the knock-out cell line can include: determining a first quantitative measurement describing the number of live wild type MCF7 breast cancer cells included in the group of wild type MCF7 breast cancer cells to which the treatment was provided; determining a second quantitative measurement describing the number of live cells included in the plurality of cells derived from the knockout cell line to which the treatment was provided.

Another embodiment of the disclosure includes a method for producing a knockout cell line from a wild type cell line. In an example embodiment, the method can include deleting a portion of the TP53 gene in a cell derived from the wild type cell line by delivering a guide RNA to the cell. Generally, the portion of the TP53 gene may include the nucleotide sequence of one or more of exons 1-11, as shown in Table 2. As an example implementation, the wild type cell line can include human MCF7 breast cancer cells and the portion of the TP53 gene can include the nucleotide sequence for exon 4.

In certain embodiments, delivering the guide RNA to the cell can include delivering an expression cassette to the cell, wherein the expression cassette includes a DNA sequence for expressing the guide RNA. In some embodiments, delivering the guide RNA to the cell further includes delivering a second expression cassette to the cell, the second expression cassette includes a DNA sequence for expressing Cas9. Without being limited to delivering the guide RNA using an expression cassette, a method for producing a knockout cell line from a wild type cell line can include providing one or more guide RNAs to the wild type cell line, the guide RNAs having the nucleotide sequences: CCATTGTTCAATATCGTCCG, GACGGAAACCGTAGCTGCCC, and TGGTTATAGGATTCAACCGG.

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIGS. 1A and 1B illustrate example genetic modifications to the TP43 gene included in certain embodiments of the disclosure.

FIG. 2 illustrates a gel in accordance with embodiments of the disclosure.

FIG. 3 illustrates a sequence comparison in accordance with embodiments of the disclosure.

FIG. 4 illustrates a gel in accordance with an embodiment of the disclosure.

FIG. 5 illustrates a sequence comparison in accordance with an embodiment of the disclosure.

FIG. 6 illustrates a sequence comparison in accordance with an embodiment of the disclosure.

FIG. 7 illustrates a gel in accordance with an embodiment of the disclosure.

FIG. 8 illustrates a graph displaying relative cell number vs. log[conc] for example knockout cell lines in accordance with an embodiment of the disclosure.

FIG. 9 illustrates a graph displaying area under the curve (AUC) for TP53 knockout (KO) pools vs. AUS TP53 for wild type (WT.)

FIGS. 10A-10D illustrate graphs displaying relative cell number vs. log[conc] for example knockout cell lines in accordance with an embodiment of the disclosure.

FIGS. 11A-11C illustrate graphs displaying a drug resistance vs. nutlin resistance. The drugs are respectively: oxaliplatin, SFU, and Palb.

Repeat use of reference characters in the present specification and drawings is intended to represent the same or analogous features or elements of the present invention.

### DETAILED DESCRIPTION

Reference now will be made to embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only and is not intended as limiting the broader aspects of the present invention, which broader aspects are embodied exemplary constructions.

The present disclosure is directed to genetically engineered cell lines which include a modification to knockout a portion of the TP53 gene. Embodiments disclosed herein provide aspects of the knockout cell line, methods for producing the knockout cell line, in vitro assays using the knockout cell line, and kits including the knockout cell line. In certain implementations, the embodiments can provide doctors and patients improved tools for determining a treatment or for comparing treatments for patients having tumors that include a TP53 mutation.

An example embodiment of the disclosure can include a knockout cell line composed of MCF7 breast cancer cells having decreased endogenous expression of at least one coding region in the TP53 gene. Generally, the cells of the knockout cell line include a genetic modification to remove or delete a portion of the TP53 gene which results in decreased endogenous expression.

In embodiments of the disclosure, the at least one coding region can include one or more of exons 4-10 in the TP53 gene. In some embodiments, the at least one coding region can include exon 4. In certain embodiments, the at least one coding region can include exon 4 and exon 5. In some embodiments, the at least one coding region can include all of exons 4-10.

For embodiments of the disclosure, the genetic modification can be applied to a native cell line (i.e., wild type). In an example implementation, the native cell line can include human MCF7 breast cancer cells, and an example embodiment can include a genetically modified MCF7 cell line having a genetic modification to remove or delete a portion of the TP53 gene. In another example implementation, the native cell line can include 600MPE, AU565, and/or BT-483. Generally, any cell line including a native TP53 gene can be genetically modified to produce a knockout cell line.

Several non-limiting examples of knockout cell lines disclosed herein include: an MCF7 cell line that includes a deletion of one or more of exons 4-10 of the TP53 gene; an AU565 cell line that includes a deletion of one or more of exons 4-5 of the TP53 gene; and a BT-483 cell line that includes a deletion of one or more exons 6-10 of the TP53 gene. These examples are provided for illustrative purposes to demonstrate how combinations of cell lines and genetic modifications may be produced using this disclosure.

Another example embodiment of the disclosure can include an in vitro assay for determining the efficacy of a treatment in cancer cells that include a TP53 gene mutation. In an example embodiment, the assay can include providing the treatment to a group of cells from a knockout cell line containing a TP53 gene mutation. In an implementation, the group of cells can be derived from any of the knockout cell lines disclosed herein (e.g., a MCF7 breast cancer cell having decreased endogenous expression of at least one coding region in the TP53 gene). During and/or after providing the treatment, the in vitro assay can further include measuring a result. In certain embodiments, measuring the result can include determining a quantitative measure of cell death (e.g., H&E staining). In some embodiments, the in vitro assay can also include providing the treatment to a group of cells from the native cell line (e.g., the MCF7 cell line). In these embodiments, the in vitro assay can also include comparing the treatment to the group of cells from the native cell line. As an example implementation, comparing the treatment to the cells from the native cell line can include determining a first quantitative measurement describing or approximating the number of live cells from the native cell line to which the treatment was provided, and determining a second quantitative measurement describing or approximating the number of live cells from the knockout cell line to which the treatment was provided. In some implementations, the first quantitative measurement and the second quantitative measurement can include a statistic, the statistic indicating if the first quantitative measurement is significantly different (e.g., higher or lower) compared to the second quantitative measurement.

For embodiments of the disclosure that include an in vitro assay, providing the treatment can include administering a drug to a group of cells from the knockout cell line. Generally, any drug can be used. Table 2 includes a list of example drugs; however, it should be understood that the list in Table 2 is not intended to be limiting and other drugs, both known and undiscovered, may be used in embodiments of the disclosure. Additionally, administering the drug can include administering one or more drugs, for example administering one or more of the drugs: Nutlin3, Fluorouracil, and Palbociclib.

A further embodiment can include a kit for assessing a treatment for a patient diagnosed with breast cancer. In an implementation, the kit can include an assay including a well-plate containing cells from a native cell line and knockout cells from a knockout cell line (the knockout cell line formed by deleting or inactivating a portion of the TP53 gene in the native cell line). In an example implementation, the cells from a native cell line can include MCF7 breast cancer cells and knockout cells can include cells derived from a knockout MCF7 cell line (including a genetic modification to one or more of exons 4-10 of the TP53 gene.) The kit can further include an indicator for measuring cell viability. In an example embodiment, the indicator can display a change in appearance (e.g., producing a color) when in contact with dead cells. Additionally, the change in appearance may be quantitative such that the intensity of the change in appearance can be related to the number of dead cells. The kit can also include the treatment (e.g., one or more drugs). In an implementation, the treatment can include Nutlin3, Fluorouracil, and Palbociclib

An additional embodiment of the disclosure includes a method for producing a knockout cell line from a native cell line, the method including deleting a portion of the TP53 gene in the native cell line. Generally, deleting a portion of the TP53 gene includes delivering a guide RNA to the native cell line and selecting for cells including the genetic modification. Example native cell lines may include: MCF7, 600MPE, AU565, and/or BT-483. Additionally, the portion of the TP53 gene can include at least one of exons 4-10.

In some implementations, delivering the guide RNA to the cell can include delivering an expression cassette to the cell, the expression cassette including a DNA sequence for expressing the guide RNA. In certain implementations, delivering the guide RNA to the cell can also include delivering a second expression cassette, including a DNA sequence expression Cas9. Several example guide RNAs for targeting the TP53 gene can include the sequences: CATTGTTCAATATCGTCCG, GACGGAAACCGTAGCTGCCC, and TGGTTATAGGATTCAACCGG.

To determine cells that have incorporated the genetic modification, a selection can be performed in some embodiments. In an example implementation, selecting for the genetically modified cell can include culturing the cells to which the guide RNA has been delivered in the presence of an agent. Exemplary agents can include any drugs to which cells derived from the native cell line are more sensitive compared to knockout cells, including a genetic modification to the TP53 gene. For example, a method for producing a TP53 knockout from the MCF7 cell line can include delivering a guide RNA to a group of cells derived from the native cell line and selecting for genetically modified cells by culturing the group of cells in the presence of Nutlin3.

Embodiments of the disclosure and examples described herein may be better understood with reference to the Sequence Listing filed with this disclosure. The Sequence Listing includes Seq ID No. 1, providing a nucleotide sequence for the TP53 gene for a Homo sapiens. Information regarding the sequence may be found from the NCBI database using the gene ID: ENSG00000141510 and transcript ID: ENST00000269305. The sequence listing also includes Seq ID No. 2 which provides an example knockout genetic sequence as observed in KO 5.6 examples. The sequence listing also includes Seq. ID No. 3 which provides an example knockout genetic sequence as observed in KO 3.4 examples.

### Example 1

Example 1 discusses various methods and provides exemplary embodiments that may be understood in conjunction with the Drawings and Description provided herein. The materials and conditions described in the example are demonstrative and are not meant to constrain the scope of the disclosure only to the materials and conditions used.

### Materials and Methods

### Cell Lines Culture

Human MCF7 breast adenocarcinoma cells (ATCC HTB-22) and their derivatives were maintained at 37° C., 5% CO2 in DMEM (Gibco, Cat. No. 11995-065) with 100 ug/mL penicillin & 100 ug/mL streptomycin (Sigma, Cat. No. P4333), 10% FBS, 50 mM Sodium pyruvate (Sigma, Cat. No. S8636), 1% GlutaMAX (ThermoFisher, Cat. No. 35050061), and 10 ug/mL insulin. MCF7 cells were passaged every 4 to 7 days to maintain sub-confluence. All cell lines were maintained in culture for a maximum of 30 passages.

Genetic Sequence TP53

The genetic sequence used for the TP53 gene is provided below using 1 letter base convention to represent the individual nucleotides (i.e., adenosine, A; guanosine, G; Cytidine, C; and thymidine, T.) The first nucleotide, base number 1, is G and the subsequent nucleotides increment by 1 base number thereon. The number appearing on the left margin represents the base number of the first nucleotide on the line. Additionally, the sequence is from a Homo sapiens (human.)

| Seq. ID No. 1: | |
|---|---|
| 1 | GTTTTCCCCT CCCATGTGCT CAAGACTGGC GCTAAAAGTT TTGAGCTTCT CAAAAGTCTA |
| 61 | GAGCCACCGT CCAGGGAGCA GGTAGCTGCT GGGCTCCGGG GACACTTTGC GTTCGGGCTG |
| 121 | GGAGCGTGCT TTCCACGACG GTGACACGCT TCCCTGGATT GGGTAAGCTC CTGACTGAAC |
| 181 | TTGATGAGTC CTCTCTGAGT CACGGGCTCT CGGCTCCGTG TATTTTCAGC TCGGGAAAAT |
| 241 | CGCTGGGGCT GGGGGTGGGG CAGTGGGGAC TTAGCGAGTT TGGGGGTGAG TGGGATGGAA |
| 301 | GCTTGGCTAG AGGGATCATC ATAGGAGTTG CATTGTTGGG AGACCTGGGT GTAGATGATG |
| 361 | GGGATGTTAG GACCATCCGA ACTCAAAGTT GAACGCCTAG GCAGAGGAGT GGAGCTTTGG |
| 421 | GGAACCTTGA GCCGGCCTAA AGCGTACTTC TTTGCACATC CACCCGGTGC TGGGCGTAGG |
| 481 | GAATCCCTGA AATAAAAGAT GCACAAAGCA TTGAGGTCTG AGACTTTTGG ATCTCGAAAC |
| 541 | ATTGAGAACT CATAGCTGTA TATTTTAGAG CCCATGGCAT CCTAGTGAAA ACTGGGGCTC |
| 601 | CATTCCGAAA TGATCATTTG GGGGTGATCC GGGGAGCCCA AGCTGCTAAG GTCCCACAAC |
| 661 | TTCCGGACCT TTGTCCTTCC TGGAGCGATC TTTCCAGGCA GCCCCCGGCT CCGCTAGATG |
| 721 | GAGAAAATCC AATTGAAGGC TGTCAGTCGT GGAAGTGAGA AGTGCTAAAC CAGGGGTTTG |
| 781 | CCCGCCAGGC CGAGGAGGAC CGTCGCAATC TGAGAGGCCC GGCAGCCCTG TTATTGTTTG |
| 841 | GCTCCACATT TACATTTCTG CCTCTTGCAG CAGCATTTCC GGTTTCTTTT TGCCGGAGCA |
| 901 | GCTCACTATT CACCCGATGA GAGGGGAGGA GAGAGAGAGA AAATGTCCTT TAGGCCGGTT |
| 961 | CCTCTTACTT GGCAGAGGGA GGCTGCTATT CTCCGCCTGC ATTTCTTTTT CTGGATTACT |
| 1021 | TAGTTATGGC CTTTGCAAAG GCAGGGGTAT TTGTTTTGAT GCAAACCTCA ATCCCTCCCC |
| 1081 | TTCTTTGAAT GGTGTGCCCC ACCCCGCGGG TCGCCTGCAA CCTAGGCGGA CGCTACCATG |
| 1141 | GCGTGAGACA GGGAGGGAAA GAAGTGTGCA GAAGGCAAGC CCGGAGGTAT TTTCAAGAAT |
| 1201 | GAGTATATCT CATCTTCCCG GAGGAAAAAA AAAAAGAATG GGTACGTCTG AGAATCAAAT |
| 1261 | TTTGAAAGAG TGCAATGATG GGTCGTTTGA TAATTTGTCG GAAAAACAAT CTACCTGTTA |
| 1321 | TCTAGCTTTG GGCTAGGCCA TTCCAGTTCC AGACGCAGGC TGAACGTCGT GAAGCGGAAG |
| 1381 | GGGCGGGCCC GCAGGCGTCC GTGTGGTCCT CCGTGCAGCC CTCCGGCCCG AGCCGGTTCT |
| 1441 | TCCTGGTAGG AGGCGGAACT CGAATTCATT TCTCCCGCTG CCCCATCTCT TAGCTCGCGG |
| 1501 | TTGTTTCATT CCGCAGTTTC TTCCCATGCA CCTGCCGCGT ACCGGCCACT TTGTGCCGTA |
| 1561 | CTTACGTCAT CTTTTTCCTA AATCGAGGTG GCATTTACAC ACAGCGCCAG TGCACACAGC |
| 1621 | AAGTGCACAG GAAGATGAGT TTTGGCCCCT AACCGCTCCG TGATGCCTAC CAAGTCACAG |
| 1681 | ACCCTTTTCA TCGTCCCAGA AACGTTTCAT CACGTCTCTT CCCAGTCGAT TCCCGACCCC |
| 1741 | ACCTTTATTT TGATCTCCAT AACCATTTTG CCTGTTGGAG AACTTCATAT AGAATGGAAT |
| 1801 | CAGGCTGGGC GCTGTGGCTC ACGCCTGCAC TTTGGGAGGC CGAGGCGGGC GGATTACTTG |
| 1861 | AGGATAGGAG TTCCAGACCA GCGTGGCCAA CGTGGTGAAT CCCCGTCTCT ACTAAAAAAT |
| 1921 | ACAAAAATTA GCTGGGCGTG GTGGGTGCCT GTAATCCCAG CTATTCGGGA GGGTGAGGCA |
| 1981 | GGAGAATCGC TTGAACCCGG GAGGCAGAGG TTGCAGTGAG CCAAGATCGT GCCACTACAC |
| 2041 | TCCAGCCTGG GCGACAAGAA CGAAACTCCG TCTCAAAAAA AAGGGGGGAA TCATACATTA |
| 2101 | TGTGCTCATT TTTGTCGGGC TTCTGTCCTT CAATGTACTG TCTGACATTC GTTCATGTTG |
| 2161 | TATATATCAG TATTTTGCTC CTTTTCATTT AGTATAGTCC ATCGATTGTA TATCCGTCCT |
| 2221 | TTTGATGGCC TTTTGAGTTG TTTCCCATTT GCGGTTATGA AATAAAGCTG CTATAAACAT |
| 2281 | TCTTGTACAA TTCTTTTTGT GATCATATGT TTTCGTGTTT CTTGGAGAAA TACTTAGGAG |
| 2341 | GGGAATTGCG AGTTTGGAAG TAAAAAGTAG CTGTATTTTG AACTTTTTCA GAAGCTCTGA |
| 2401 | GTTTTCCAGA GCGGTTGTAC CATTTTACAC TCCAACTAGC AAGGTATGGG AGTTATTATG |
| 2461 | GTTGTGCCAC AGCCTTCCGG ACATTAGGTA TTGTCAGTCT TTCTAATGTG GTATATCCTT |
| 2521 | GTGGTTGTAA TTTACAGTTC TCTATTGACT AAGGATGTTC AGCATTTTTT CATGTGCCTA |
| 2581 | TTGGCCATTC GTATTTTGTT TGTAAAGTAG CTCTTCGAGT CTTTTACCTG TTATTTTGGT |
| 2641 | TTTTTGTTTG TTTTTATTGT TCAGTTGTGG GACTGCTTTA TACATTCTGG ATACAAGTCC |
| 2701 | TTTATCAGAT CCATGTGTCG TGAATGTTTT CTTCTGATCT GTTGCTTGCC TATTTGTTTG |
| 2761 | CTTTACAGAG TTTACAGTAT CTTAAGAGGA GTGGATTTAT CTTTTTTATG TTCAGTATTT |
| 2821 | GCCTTGTCCT GTTTAGGACA TCTTTTTTTT TTTTTTTAAC CCCAGGGTCA TGAAGATATT |
| 2881 | ATCTTACATT TTCTTTTAGG ACCTTTATGG TTGTAAGTTT TACAGTAAGG TCCTTGAGCC |
| 2941 | ATTAATTAAT TCTTAAAATT AATTGTTTAT GGTGTGAGGT GTAGGAGTCA GTCTCTGGTA |
| 3001 | TCTTTCCTGT ATGGAAATCC AGTTATTCTG TCTCCACTTG TTGAAATAGG CTTCCTTTCT |
| 3061 | CTACTGAATG CTTTTAATTT TAATTATTTT ACAGTTGGAG TATAGGGCTA CCATTTTAGT |
| 3121 | GCTATTTTCT TTTTTTCTTT GTTAATTTTT GAGACAGGGA CTCACACTGT TGCCCAGGCT |
| 3181 | AGAGTACAAT GGCACAATCA AGGCTTACTG CAGCCTCGAA CCCCTGGGCT CAAGCAGTCC |
| 3241 | TCTAGCAGCC TCACGAGTAG CTGGGATTAC TCCACCACAC CCAGCTAACT ATTTTATTTT |
| 3301 | TTTGTATTGA CAGGATCTCA CTATGTTGCC CAGGCTGGTC TCAAACTGCT GGCCTCAAGC |
| 3361 | TTTCATCCCA TCTCGGCCTC CCAAAGTGCT GGGATTACAG GTGTGAGCCA CCATGCCTGA |
| 3421 | CCTCTTAGTG CTATTTTCTA TTTATCTCCT CTGTTCTCTG CTCTCTTTAA ACGTTGGAGG |
| 3481 | AAGAAACAGT ACCCATCTTA CACAAACTCT TCAGAAAACA GAGGAACAGA CTGGGCGCGG |
| 3541 | TGGCTCATAC CTGTAATCTC AGCACTTTGG TACGCTGAGG CAGGGGATCA TTTGAGGTCG |
| 3601 | GGAGTTCGAG ACCAGCCTGG CCAACACGGC GAAACCCCAT CTCTACTAAA AATACAAAAA |
| 3661 | GTAGCTAGGC GTGGTGACAC ATACCTGTAA TGCCAGTTAC TCAGGAGGCT GAGGCACAAG |
| 3721 | AATCCCTTGA ACCTGGGAAG CGGAGGTTGC AGTGAGCCGA GATTGCGCCA CTGCACTCCA |
| 3781 | GCCTGGGCAA CAGAGTGAGA CCCTGTCTCA GAAAAAAAAA GAAAGAAAGA AAAAATAGAG |
| 3841 | GAATATTTCC CAACTTGTTT TCGAAGCCAG CATAATCCTG GTACCAAAAC CAAACAAGGA |
| 3901 | CATTATAAGA AAAGAAAATA TAGACCAATA TTCCTGTTAG CATAGACATG CAACAGCTAA |
| 3961 | CCAATTTTAG CAAACCAAAC CTGGTAATAT AGAAAAAAGG ATAAATAGGC CAGTCGCGGT |
| 4021 | GGCTCACGCC TGTAATCCCA GCACTTTGGG AGGCTGAGGC AGGCAGATCA CTTGAGGTCA |
| 4081 | GGAGTTTGAG ACCAGCCTGA CCAACATGGT GAAACCCCGT TTCTAATAAA AATACAAAAA |
| 4141 | TCAGGCTGGG CACGGTGGCT CACGCCTGTA ATCCCAGCAC TTTGGGAGGC CGAGGTGGGC |
| 4201 | AGATCACGAG GTCAGGAGTT CAAGACCAGC CTGACCAATG TGGTGAAACG CCATCTCTAC |
| 4261 | TAAAAATACA AAAATCAGCC GGTGTGGTGG CACCTGCCTG TAATCCCAGC TACTCAGGAG |
| 4321 | GCTGAGGCAG AATTGCTTGA ACCCGGGAGG CAGAGGTTGC AGTGAGCCAA GATCGTGCCA |
| 4381 | CTGCACTCCA GCCTGGGCGA CAGAGCAAGA CTTCATCTCA AAAAAAAAAA AAAATTAGCT |
| 4441 | GGGCATGGTG GTGGGCACCT GAAATCCCAG CTACTCGGGA GTCTGAGGCA GGAGAATCGC |
| 4501 | TTGAACCCAG GAGGCAGAAG TTGCACTGAG CTGGGATCAC ACCATTGCAC TCCAGCCTGG |
| 4561 | GCAACAGAGT GAGACTCCAT CTCAAAAAAA GAAAAAGAAA AAGGATAAAT ACATTCTAAC |
| 4621 | CAAATAATGT TTATCTCATG ATTGTAGCTG ATTCAACATT CAAAAATTGG CCTGGTGCAG |
| 4681 | TAGCTCAGGC CTGTAATCCC AACATTTTAG GAGGCTGAGG CAGGAAGATC TCTTGAGCCC |
| 4741 | AGGATTTCAA GACCAGCCTG GGCAACATAG TCAGACTGGT CTTTACTGGG GGGAAAAAAA |
| 4801 | TCAGTCTGTG TAATTCACCA CATTAACAAA GGGAAACATA AAAACCCTAT GATCATTTCA |
| 4861 | ACAGATGTAG CAAAAGCAGT TAATGATATT CAACACATAT GCATGATTAC AAACCAACCA |
| 4921 | ACCTCCTAGC AAACTAGGGA AAGGAAACTT AACCTAGTTT GATAACAGGG CGTCCACAGT |
| 4981 | CGGAGTTCCA CTAGCAGCAT ACATAATGGT AGAAAACTCA GTGCTGCCGG GCGCGGTGGC |
| 5041 | TCACGCCTGT AATGCCAGCA CTTTGGGAGG CCTAGGCGGG CGGATCACGA GGTCAGGAGA |
| 5101 | TCGAGACTGT CCTGACTAGC ATGCTGAAAC CCCGTCTCTA CTAAAAATAC AAAAACAAAA |
| 5161 | AATTAGCCGG GCATGGTGGC GGGCGCCTAT AGTCCCAGCT ACTCGGGAGG CTGAGGCGAG |
| 5221 | AGAATGGCGT GAACCCGGGA GGCGGAGCTT GCAGAGCCTA GATCGTGCCA CTGCACTCCA |
| 5281 | GCCTGGGTGA CAGAGTGAGA CTTCGTCTCA AAAAAAAAAA AGAAAAGAAA |
| 5341 | ACTCAACGCT TTTTCCTCTA AGATCAGGAA CTAGAAAAGG ATTTGACTCT CACAACGTTG |
| 5401 | ATACCATACT GGAGGTTTTA ACCAGGCAAG AAAAAGAAAT AATGAGGGCC GGGTGCGGTG |
| 5461 | GCTCAGGCCT GTAATCCCAG CACTTTGGGA AGCCGAGACG GGTGGATCAC GAGGTCAGGA |
| 5521 | GATCGAGACC ATCCTGGCTA ACACGGTGAA ACCCTGTCTC TACTAAATAT ACAAAAAATT |
| 5581 | AGCCGGGCGT AGTGGCGGGC GCCTGTAGTC CCAGCTACTC GGGAGGCTGA GGCAGGAGAA |
| 5641 | TGGCGTGAAC TCAGGGGGCG GAGCTTGCAG TGAGCTGAGA TCGAGCCACT GCACTCCAGC |
| 5701 | CTGGGCGACA GAGCAAGACT GTGTCTCAAA AAAAAAAAAA GAAAAAGAAA TAATGATTAG |
| 5761 | TGGCCCGATG TCTCACGCCT ATAATCCCAG CACTTTGGGA GGCCGAGGTG GGCAGATCAC |
| 5821 | CTGAGGTCTG GAGTTGGAGA CCAGCCTGAC AAAGATGGTG AAACCTCGTC TCTATTAAAA |
| 5881 | TATTAAAAAA ATAGCCAGGC GTTGGCCGGG TACAGTGGCT CATGCCTGTA ATCCCAGCAC |
| 5941 | TTTGGGAGGC CGAGGTGGGT GGATCACCTG AGGTCAGGAG TTCAACACCA GCCTGGCCAA |
| 6001 | CATGGTGAAA CCCCATCTCT ACTAAAAATA CAAAAATTAG CCGGGCGTAG TGGCGGGCGC |
| 6061 | CTGTAATCCC AGCTACTTGG GAGGCTTAGG CAGGAGAATC GCTTGAACCT GGGAGGCGGA |
| 6121 | GGTTGTAGTG AGCCGAGATT GCACCATTGC ACTCCAGCCT GGGTGACAAA AGCAAAAACT |
| 6181 | CCGTCTCAAA AAAAAAAGAA TTAGCCAGGG GTAGTGGTGA ACGCCTGTAG TCCCAGCTAC |
| 6241 | TCAGGAGGCA GAGGCAGGAG AATCACTTGA ACCCAGGAGG CAGAGGTTGC AGTGAGCCGA |
| 6301 | GATTGTCCCA TTGCACTCCA GCCTAGGCGA CAAGAGCAAA ATTCCATGTC AAAAAAAAAA |
| 6361 | AAAAAAAAGG AAAGAAAAAA AATAACGATT AGAAAGGAAG AAATAAAACA CATTCACAGC |
| 6421 | CAGTATGATT CTATACATAC ATGTCCTAAT GGGGCCAGGC GTGGTGGCTC ATGCCTGTAA |
| 6481 | TCCTAGCACT TTTAGGAGGC TGAGGCAGGT GGCTTCCCTG GGACCAGCCT GGCCAACATG |
| 6541 | GTGAAACCCC AACTCTAATA AAAATACAAA AAATCAGCCA GGCGTGGTGA CGGGCACCTC |
| 6601 | TAATCCCAGC TACTCAGGAG GCTGAGGCAG GAGAATTGCT TGGACCTGGG AGGCAGAGGT |
| 6661 | TGCAGTGAGC CGAGATCGCG CTATTGCACT CCAGCCTGGG CAACAAGAGT GAAACTCCGG |
| 6721 | CAGGGTGTGG TGGCTTACGC CTGTAATCCC AGCACTTCGG GAGGCTGAGG CAGGCCGATC |
| 6781 | ACCTGAGGTC AGGAGTTTGA GACCAACCTA ACATGGTGAA ACCCCGTCTC TACTAAAAAT |
| 6841 | ACAAGAATTA GCTGGGTGTA GTGGTGGGCG CCTGTAATCC CAGCTACTTG GGAGGCTGAG |
| 6901 | ACAGAAGAAT TGCTTGAACC CAGGAGGTGG AGGTTGCAGT GAGCTGAGAT CATGCCATTG |
| 6961 | CACACCACGC CGGGCAACAG AGCGAGATTC CGTCTCAAAA AAAAAAAAAA AGAGTGAAAC |
| 7021 | TCTATCTCAA AAAAAAAAAA AAGTCCTAAT GGAAAATCCA TAAAAAGCTA CCAAAACTAA |
| 7081 | TAAATAAATA TAGCAGGGTT GCAGGTTACA GGGCAATATA GTTATCCCTC TATCTGTAGG |
| 7141 | GGCTTGGTTC TGGGACTCCT CACACACCAA ACCCACAGAT GTCTAAGTCC CATATATAAG |
| 7201 | ACGGTATAGT ATTTGGATTT AACCTACACA TATCCTCCCA TATAGTTTAA ATTATCTCTA |
| 7261 | GATTACTTAC ATTACCCCCA TACAATGAAA ATGCTAATGT ACATGCAAGT ATGTATGTAA |
| 7321 | GTACTTGTAC TATATTGTTT AGGGAATCAC TGGACATATA GGCCTTCAAG ACTGATACCA |
| 7381 | GCAGCCACTG TTAAGATTCT GGTCAGGCCT GCCCCTGTTT GGGGTCTCAG TTGATCTCAT |
| 7441 | TGCCTTCCCA CCCAGCCAAG GGCACCTGCA TTTCTCTTGG CTCCCTGGCC ATTTGGAAGG |
| 7501 | CCTAGTTCAG CCTGGCACAT TTGTATCCTG GCCCACTGAT GCTGGTACCC CTGGGAAGGT |
| 7561 | CCTGCTCTGA AAAACACGGA GATTTTAGTT GCTACTGAAG ATTTGAGAGA TAAAGACAGG |
| 7621 | GAGACCTGTC TGTAGACCTG TGTCCCTCCA AGTGGGATTG AGACTTTGGG CCCCCCATTT |
| 7681 | CAGGACAGCA CCTCCTGGCC TGTTGACTGA ATAGATCCCT GAAGGAGGTG TACTTGCATT |
| 7741 | AATGGAGTGG GGGTGGGAGC AGTACCACAG ATCCGCACTA ACAATCACAC AGTTCTCTCT |
| 7801 | AGAATAATAA TATAGAACAA GTGAAATAGA ACAATTGCAG AAAGAGCTAA CCTTTGTTGA |
| 7861 | GCTCTTACTG TGTGCCCAGC ACTTTCCTCA ACTCTACATT TCCCATAATA CACAGAGTAC |
| 7921 | TAGGTAGGCC AGGCTTGGTG GCTCACGCCT GTAATCCCAG CACTTTAGGA GGCCAAGGGG |
| 7981 | GGTGGATCAC CTGAGGTCGG GAGTTCAAGA CCAGCCTGAC CAACATGGTG AAACCCCGTC |
| 8041 | TCTACTAGAA GTACAAAATT AGCCAGGTGT GGTGGCACAT GCTTGTAGTC CTAGCTACTC |
| 8101 | AGCAGGCTGA GGCAGGAGAA TCATTTGAAT CCGGGAGGAG GTTGCAGTAA GCGGAGATAG |
| 8161 | TGCCACTGTA CTCCAGCCTG GGCAATAAGA GCTGAGACTC CGTCTCAAAA TAAAATAAAA |
| 8221 | TAAAATAAAA AAAGAAAAGA GCCTGCCATT AAAGGAGCTG |
| 8281 | TTTGGTAGGG GATGTTTTGT CAGTGCAAAC AACAGAAAAG TGGGCTGGGC ACAGTGGTTC |
| 8341 | ATGCCTGTAA TCCCAGCACT TTGGGAGGCC AAGGCGGGCG GATCACCTGA AGTTGGGAGT |
| 8401 | TCAAGACCAG CCTGACCAAT ATGGAGAAAC CCCGTCTCTA CTAAAAATAC AAAATTAGCC |
| 8461 | GGGCGCAGTG GCGCATGCCT GTAATCCCAG CTACTCGGGA GGCTGAGGCA GGAGAATCGC |
| 8521 | TTGAACCTGG GAGGCAGAGG TTGCGGTGAG CCGAGATCGC ACCATTGCAC TCCAGCCTGG |
| 8581 | ACGAGAGCAA AACTCTGTCT CAAAAAAAAA AAAAAACAGA AAAGTGTAAC AAACACTTAC |
| 8641 | AGTAGGCATG TTTCTTAGCA AATCTGATGA CAAATTTGGC ATAAAGAAAG AGAGCATCCC |
| 8701 | TGAAAAAAAA AAAAAGAAAA AGAAAGAGAG CATCCTGCCT GGGCAACATA GTGAAACCCT |
| 8761 | GCCTCTACAA AAAAACTCAA AAATTGGCCG GGTGCAGTGG CTCACACCTG TAATCCCAGC |
| 8821 | ACTTTGGGAG TCGGAGGCGG GAGGATCACC TGAGGTCAGG AGTTCGAAAC CAGCCTGGCC |
| 8881 | AACATGGCAA AACCCCATCT CTACTAAAAA TACAAAAAAT TAATCAGGCG CATTGGTGGG |
| 8941 | CGCCTGTAAT CCCAGCTACT CAGGAAGTTG AGGCAAGAGG ATCGCTTGAA TCTGGGAGGT |
| 9001 | GGAGGTTACA GTGAGTCGAG ATCACACCAC TGCACTCTAG CCTGGGTGAC AGGGCGAGAC |
| 9061 | TCCGTCTCCA AAAAAAAAAA GAAAAAGAAA AAGACTAAAA AATTAGCCAG GCAGGCCTCT |
| 9121 | GTGGTCCCAG CTACTTGGGA GGCTGAGGCA GGAGAATCAC TGAGCCCAGG AGTCCGAGGC |
| 9181 | TGTAGTGAGC CATGATTGCA CCACTGTACC CTAGCTTGGG CAACAAAGCA AGACCCTGCC |
| 9241 | TCAAAAGAAA AAAGAAAGAA AGAAAGAACA TGGCGGGCCA GGCACAGTGG CTCACACCTG |
| 9301 | TAATCCCAGC GCTTTGAGAG GCCGAGGCAG GTGGATCACA AGGTCAGGAG TTCCACACCA |
| 9361 | GCCTGGCCAA CATGGTGAAA CCCTGTCTCT ACTAAAAATA CAAAAAATCA GCCAGGCATG |
| 9421 | GTGGCAGGGG CCTGTAATCC CAGCTACTCG GGAGGCTGAG GCAGGAGAAT TGCTTGAAAC |
| 9481 | CAGAAGGCAG AGGTTGCAGT GAGCCTAGAC TGCACCACTG CACTCCAGCC TGGGCGAAAA |
| 9541 | GAGCCAAACT CCATCTCAAA AAACAAACAA AAAAACAAAA CAAAAGAAAA CATGGCAAAG |
| 9601 | CCTTTGAAAG CTTGTCTGGG AGAAGGTGCG ATGATAGTTG CATAACTTCG TGCAAGATGC |
| 9661 | TGGTCCACAC AGGGGCTGCC CCTTGCTCTT TCTCGCTCTC TTAACCTCTC ATATAACAGG |
| 9721 | CTTGTGTGTT ATTCACATTT ATTGAGCCCA AGCAGGTGCA AGGCATTGTG ATCTAATACT |
| 9781 | TTGGTCAGCA AGACAACAAG ATAGATCACT GCCCTGCCCT TAGGAAGTGT ATATGCTATT |
| 9841 | AGAGGAAACA GATAAAATAA ACAAGGAAAA GTATCAGACA ATGTAAGTGC TATGAGAATG |
| 9901 | CAAATGAGGT GATGTGAATT AAAATAGGAT GACTTAAAGT CTGCACGGGA AGGAGCCTAC |
| 9961 | CCCCATGTTC CTGGCTAGCC AAGGAACCAC CAGTTGATTA GCAGAGAAGG GCAGCCAGTC |
| 10021 | TAGCTAGAGC TTTTGGGGAA GAGGGAGTGG TTGTTAAGAG ATGAGATTAA AGAAGCCGAG |
| 10081 | ACGGGCCATT CGTGAGGGGT TTGTAATGCA GGGCTGAGGA GTGTCCGAAG AGAATGGGCA |
| 10141 | GGTGAGCGGT GAGACAGTTG TTCTTCCAGA AGCTTTGCAG TGAAAGGAAT CAAAGAAATG |
| 10201 | GAGCCGTGTA TCAGGTGGGG AAGGGTGGGG GCCAAGGGGG TGTCCTTCCC CATACAGAGA |
| 10261 | TTGCAGGCTG AGAATGACTA TATCCTTGTT AACAGGAGGT GGGAGCAGGG CACGGTAGCT |
| 10321 | CACACCTGTA ATCTTGGCAC TTTAGGAGGC TGAGGCGGGC CGATCACCTG AAGTAAGGAG |
| 10381 | TTCGAGACCA GCCTGGCCAA CATGCAAAGC CCTGTCTCTA CTAAAAATAC AAAAATTAGC |
| 10441 | TGGGTGTGGT GGTACTCGCC TGTAATCCCA GCTACTCGGG AGACTGAGGC AGGAGAATGG |
| 10501 | CTTGAACCCG GAAGGTAGAG GTTGCAGTGA GCTGAGATCA TGCCACTGTG CTCCAGCCTA |
| 10561 | GGTGACAGAG AGAGACTCCA TCTCAAAAAA AAAAAAAAAA TACAGGAAGG GAGTTGGGAA |
| 10621 | TAGGGTGCAC ATTTAGGAAG TCTTGGGGAT TTAGTGGTGG GAAGGTTGGA AGTCCCTCTC |
| 10681 | TGATTGTCTT TTCCTCAAAG AAGTGCATGG CTGGTGAGGG GTGGGGCAGG AGTGCTTGGG |
| 10741 | TTGTGGTGAA ACATTGGAAG AGAGAATGTG AAGCAGCCAT TCTTTTCCTG CTCCACAGGA |
| 10801 | AGCCGAGCTG TCTCAGACAC TGGCATGGTG TTGGGGGAGG GGGTTCCTTC TCTGCAGGCC |
| 10861 | CAGGTGACCC AGGGTTGGAA GTGTCTCATG CTGGATCCCC ACTTTTCCTC TTGCAGCAGC |
| 10921 | CAGACTGCCT TCCGGGTCAC TGCCATGGAG GAGCCGCAGT CAGATCCTAG CGTCGAGCCC |
| 10981 | CCTCTGAGTC AGGAAACATT TTCAGACCTA TGGAAACTGT GAGTGGATCC ATTGGAAGGG |
| 11041 | CAGGCCCACC ACCCCCACCC CAACCCCAGC CCCCTAGCAG AGACCTGTGG GAAGCGAAAA |
| 11101 | TTCCATGGGA CTGACTTTCT GCTCTTGTCT TTCAGACTTC CTGAAAACAA CGTTCTGGTA |
| 11161 | AGGACAAGGG TTGGGCTGGG GACCTGGAGG GCTGGGGACC TGGAGGGCTG GGGGGCTGGG |
| 11221 | GGGCTGAGGA CCTGGTCCTC TGACTGCTCT TTTCACCCAT CTACAGTCCC CCTTGCCGTC |
| 11281 | CCAAGCAATG GATGATTTGA TGCTGTCCCC GGACGATATT GAACAATGGT TCACTGAAGA |
| 11341 | CCCAGGTCCA GATGAAGCTC CCAGAATGCC AGAGGCTGCT CCCCCCGTGG CCCCTGCACC |
| 11401 | AGCAGCTCCT ACACCGGCGG CCCCTGCACC AGCCCCCTCC TGGCCCCTGT CATCTTCTGT |
| 11461 | CCCTTCCCAG AAAACCTACC AGGGCAGCTA CGGTTTCCGT CTGGGCTTCT TGCATTCTGG |
| 11521 | GACAGCCAAG TCTGTGACTT GCACGGTCAG TTGCCCTGAG GGGCTGGCTT CCATGAGACT |
| 11581 | TCAATGCCTG GCCGTATCCC CCTGCATTTC TTTTGTTTGG AACTTTGGGA TTCCTCTTCA |
| 11641 | CCCTTTGGCT TCCTGTCAGT GTTTTTTTAT AGTTTACCCA CTTAATGTGT GATCTCTGAC |
| 11701 | TCCTGTCCCA AAGTTGAATA TTCCCCCCTT GAATTTGGGC TTTTATCCAT CCCATCACAC |
| 11761 | CCTCAGCATC TCTCCTGGGG ATGCAGAACT TTTCTTTTTC TTCATCCACG TGTATTCCTT |
| 11821 | GGCTTTTGAA AATAAGCTCC TGACCAGGCT TGGTGGCTCA CACCTGCAAT CCCAGCACTC |
| 11881 | TCAAAGAGGC CAAGGCAGGC AGATCACCTG AGCCCAGGAG TTCAAGACCA GCCTGGGTAA |
| 11941 | CATGATGAAA CCTCGTCTCT ACAAAAAAAT ACAAAAAATT AGCCAGGCAT GGTGGTGCAC |
| 12001 | ACCTATAGTC CCAGCCACTT AGGAGGCTGA GGTGGGAAGA TCACTTGAGG CCAGGAGATG |
| 12061 | GAGGCTGCAG TGAGCTGTGA TCACACCACT GTGCTCCAGC CTGAGTGACA GAGCAAGACC |
| 12121 | CTATCTCAAA AAAAAAAAAA AAAAAGAAAA GCTCCTGAGG TGTAGACGCC AACTCTCTCT |
| 12181 | AGCTCGCTAG TGGGTTGCAG GAGGTGCTTA CGCATGTTTG TTTCTTTGCT GCCGTCTTCC |
| 12241 | AGTTGCTTTA TCTGTTCACT TGTGCCCTGA CTTTCAACTC TGTCTCCTTC CTCTTCCTAC |
| 12301 | AGTACTCCCC TGCCCTCAAC AAGATGTTTT GCCAACTGGC CAAGACCTGC CCTGTGCAGC |
| 12361 | TGTGGGTTGA TTCCACACCC CCGCCCGGCA CCCGCGTCCG CGCCATGGCC ATCTACAAGC |
| 12421 | AGTCACAGCA CATGACGGAG GTTGTGAGGC GCTGCCCCCA CCATGAGCGC TGCTCAGATA |
| 12481 | GCGATGGTGA GCAGCTGGGG CTGGAGAGAC GACAGGGCTG GTTGCCCAGG GTCCCCAGGC |
| 12541 | CTCTGATTCC TCACTGATTG CTCTTAGGTC TGGCCCCTCC TCAGCATCTT ATCCGAGTGG |
| 12601 | AAGGAAATTT GCGTGTGGAG TATTTGGATG ACAGAAACAC TTTTCGACAT AGTGTGGTGG |
| 12661 | TGCCCTATGA GCCGCCTGAG GTCTGGTTTG CAACTGGGGT CTCTGGGAGG AGGGGTTAAG |
| 12721 | GGTGGTTGTC AGTGGCCCTC CAGGTGAGCA GTAGGGGGGC TTTCTCCTGC TGCTTATTTG |
| 12781 | ACCTCCCTAT AACCCCATGA GATGTGCAAA GTAAATGGGT TTAACTATTG CACAGTTGAA |
| 12841 | AAAACTGAAG CTTACAGAGG CTAAGGGCCT CCCCTGCTTG GCTGGGCGCA GTGGCTCATG |
| 12901 | CCTGTAATCC CAGCACTTTG GGAGGCCAAG GCAGGCGGAT CACGAGGTTG GGAGATCGAG |
| 12961 | ACCATCCTGG CTAACGGTGA AACCCCGTCT CTACTGAAAA ATACAAAAAA AAATTAGCCG |
| 13021 | GGCGTGGTGC TGGGCACCTG TAGTCCCAGC TACTCGGGAG GCTGAGGAAG GAGAATGGCG |
| 13081 | TGAACCTGGG CGGTGGAGCT TGCAGTGAGC TGAGATCACG CCACTGCACT CCAGCCTGGG |
| 13141 | CGACAGAGCG AGATTCCATC TCAAAAAAAA AAAAAAAAGG CCTCCCCTGC TTGCCACAGG |
| 13201 | TCTCCCCAAG GCGCACTGGC CTCATCTTGG GCCTGTGTTA TCTCCTAGGT TGGCTCTGAC |
| 13261 | TGTACCACCA TCCACTACAA CTACATGTGT AACAGTTCCT GCATGGGCGG CATGAACCGG |
| 13321 | AGGCCCATCC TCACCATCAT CACACTGGAA GACTCCAGGT CAGGAGCCAC TTGCCACCCT |
| 13381 | GCACACTGGC CTGCTGTGCC CCAGCCTCTG CTTGCCTCTG ACCCCTGGGC CCACCTCTTA |
| 13441 | CCGATTTCTT CCATACTACT ACCCATCCAC CTCTCATCAC ATCCCCGGCG GGGAATCTCC |
| 13501 | TTACTGCTCC CACTCAGTTT TCTTTTCTCT GGCTTTGGGA CCTCTTAACC TGTGGCTTCT |
| 13561 | CCTCCACCTA CCTGGAGCTG GAGCTTAGGC TCCAGAAAGG ACAAGGGTGG TTGGGAGTAG |
| 13621 | ATGGAGCCTG GTTTTTTAAA TGGGACAGGT AGGACCTGAT TTCCTTACTG CCTCTTGCTT |
| 13681 | CTCTTTTCCT ATCCTGAGTA GTGGTAATCT ACTGGGACGG AACAGCTTTG AGGTGCGTGT |
| 13741 | TTGTGCCTGT CCTGGGAGAG ACCGGCGCAC AGAGGAAGAG AATCTCCGCA AGAAAGGGGA |
| 13801 | GCCTCACCAC GAGCTGCCCC CAGGGAGCAC TAAGCGAGGT AAGCAAGCAG GACAAGAAGC |
| 13861 | GGTGGAGGAG ACCAAGGGTG CAGTTATGCC TCAGATTCAC TTTTATCACC TTTCCTTGCC |
| 13921 | TCTTTCCTAG CACTGCCCAA CAACACCAGC TCCTCTCCCC AGCCAAAGAA GAAACCACTG |
| 13981 | GATGGAGAAT ATTTCACCCT TCAGGTACTA AGTCTTGGGA CCTCTTATCA AGTGGAAAGT |
| 14041 | TTCCAGTCTA ACACTCAAAA TGCCGTTTTC TTCTTGACTG TTTTACCTGC AATTGGGGCA |
| 14101 | TTTGCCATCA GGGGGCAGTG ATGCCTCAAA GACAATGGCT CCTGGTTGTA GCTAACTAAC |
| 14161 | TTCAGAACAC CAACTTATAC CATAATATAT ATTTTAAAGG ACCAGACCAG CTTTCAAAAA |
| 14221 | GAAAATTGTT AAAGAGAGCA TGAAAATGGT TCTATGACTT TGCCTGATAC AGATGCTACT |
| 14281 | TGACTTACGA TGGTGTTACT TCCTGATAAA CTCGTCGTAA GTTGAAAATA TTGTAAGTTG |
| 14341 | AAAATGGATT TAATACACCT AATCTAAGGA ACATCATAGC TTAGCCTAGC CTGCTTTTTT |
| 14401 | TTTTTTTTTT TTTGGAGACA GAGTCTCACT CTGTCACCCA GGCTGGAGTG CAGTGGCGGG |
| 14461 | ATCTCGGCTC ACTGCAACCT CCGCCTTCTG GGTTCAAGCG ATTCTCCTGC CTCAGCCCAC |
| 14521 | TGAGTAGCTG GGATTACAGG CACCTGCCCC GACGCCCAGC TAATTTTTTG TTATTTATTT |
| 14581 | ATTTTTTTTT TTAGTAGAGA TGAGGTTTCA CCATGTTGGC CAGGCTAGTC TCGAACTCCT |
| 14641 | GACCTTGTGA TCTGCCTGCC TTGGCCTCCC AAAGTGCTGG GATTACAGGC GTGAGCCACC |
| 14701 | GCACCCGGCC TGCCTAGCCT ACTTTTATTT TATTTTTAAT GGAGACAGCA TCTTGCTCTG |
| 14761 | TTGCCCAGGC TGGATTACAG TGATGTGATC ATAGCTCATT ATACCCTCCT GGGCTCAAGC |
| 14821 | AATCCCCCTA ACTCTGCCTC CCCAGTAGCT AGGACCACAG GCATACACCA CCATACCCAG |
| 14881 | CTAATTTTTA AAATTTTTTG TAGATAGATA GAGTCTCACT ATGTTGCCCA GGCTGGTCTC |
| 14941 | TAGCCTACTT TTTTGAGACA AGGTCTTGCT CTGTCACCCA GGCTGGATAG AGTGCAGTAG |
| 15001 | TGCAGTCACA GCTCACTGCA GCCTCCACCT CCCAGGCTCC ATCCATCCTC CCAGCTCAGC |
| 15061 | CTCCCAAGTT GCTTCAACTA CAGGCCTGCA CCACCATGCC TGGCTAATTT TTATTTATTT |
| 15121 | ATTTTTATTT TATTTTATTT TATTTTTTTG AGACTCAGTC TCACTCTGTC GCCCAGGCTG |
| 15181 | GAGTGCAGTG GCATGATCTC GGCTCACTGC AACCTCTGCC TCCTGGGTTC AAGTGATTCT |
| 15241 | CCTGCCTCAG CCTCCCGAAT AGCTAGGACT ACAAGCGCCT GCTACCACGC CCAGCTAATT |
| 15301 | TTTGTATTTT TAGTAGAGAC AGGGTTTCAC CATGTTGGCC AGGCTGGTCT CGAACTTCTG |
| 15361 | ACCATGTGAT CCGCCCGCCT CGGCCTCCCA AAGTGCTGGG ATTACAGGTG TGAGCCACCA |
| 15421 | CGCCCGGCTA ATTTTTATTT ATTTATTTAA AGACAGAGTC TCACTCTGTC ACTCAGGCTA |
| 15481 | GAGTGCAGTG GCACCATCTC AGCTCACTGC AGCCTTGACC TCCCTGGGCT CCGGTGATTT |
| 15541 | CACCCTCCCA AGTAGCTAGG ACTACAGGCA CATGCCACGA CACCCAGCTA ATTTTTTATT |
| 15601 | TTCTGTGAAG TCAAGGTCTT GCTACGTTGC CCATGCTGGT ATCAAACCCC TGGGCTCAAT |
| 15661 | CAATCCTTCC ACCTCAGCCT CCCCAAGTAT TGGGGTTACA GGCATGAGCT ACCACACTCA |
| 15721 | GCCCTAGCCT ACTTGAAACG TGTTCAGAGC ATTTAAGTTA CCCTACAGTT GGGCAAAGTC |
| 15781 | ATCTAACACA AAGCCCTTTT TATAGTAATA AAATGTTGTA TATCTCATGT GATTTATTGA |
| 15841 | ATATTGTTAC TGAAAGTGAG AAACAGCATG GTTGCATGAA AGGAGGCACA GTCGAGCCAG |
| 15901 | GCACAGCCTG GGCGCAGAGC GAGACTCAAA AAAAGAAAAG GCCAGGCGCA CTGGCTCACG |
| 15961 | CCTGTAATCC CAGCATTTCG GGAGGCTGAG GCGGGTGGAT CACCTGAGGT CAGGAGTTCA |
| 16021 | AGACCAGCCT AGCCAACATG GTGAAACCCC GTCTCTACTA AAATACAAAA ATTAACCGGG |
| 16081 | CGTGATGGCA GGTGCCTGTA ATCCCAGCTA CTTGGGAGGC TGAGGCAGGA GAATCGCTTG |
| 16141 | AACCAGGAGG CGGAGGTTGC AGGGAGCCAA GATGGCGCCA CTGCACTCCA GCCTGGGCGA |
| 16201 | TAGAGTGAGA CTCCGTCTCA GAAAAAAAAG AAAAGAAACG AGGCACAGTC GCATGCACAT |
| 16261 | GTAGTCCCAG TTACTTGAGA GGCTAAGGCA GGAGGATCTC TTGAGCCCAA GAGTTTGAGT |
| 16321 | CCAGCCTGAA CAACATAGCA AGACATCATC TCTAAAATTT AAAAAAGGGC CGGGCACAGT |
| 16381 | GGCTCACACC TGTAATCCCA GCACTTTGGG AGGTGGAGGT GGGTAGATCA CCTGACGTCA |
| 16441 | GGAGTTGGAA ACCAGCCTGG CTAACATGGT GAAGCCCCAT CTCTACTAAA AACACAAAAA |
| 16501 | TTAGCCAGGT GTGGTAGCAC ACGCCTGTAG TCCCAGCTAC TCGGGAGGCT GAGGCACAAG |
| 16561 | AATCACTTGA ACCCCAGAGG CGGAGATTGC AATCAGCCAA GATTGCACCA TTGCACTCCC |
| 16621 | GCCTGGGCAA CAGAGTGAGA CCCCATCTCA AAATAAATAA ATAAATATTT TTAAAAGTCA |
| 16681 | GCTGTATAGG TACTTGAAGT GCAGTTTCTA CTAAATGCAT GTTGCTTTTG TACCGTCATA |
| 16741 | AAGTCAAACA ATTGTAACTT GAACCATCTT TTAACTCAGG TACTGTGTAT ATACTTACTT |
| 16801 | CTCCCCCTCC TCTGTTGCTG CAGATCCGTG GGCGTGAGCG CTTCGAGATG TTCCGAGAGC |
| 16861 | TGAATGAGGC CTTGGAACTC AAGGATGCCC AGGCTGGGAA GGAGCCAGGG GGGAGCAGGG |
| 16921 | CTCACTCCAG GTGAGTGACC TCAGCCCCTT CCTGGCCCTA CTCCCCTGCC TTCCTAGGTT |
| 16981 | GGAAAGCCAT AGGATTCCAT TCTCATCCTG CCTTCATGGT CAAAGGCAGC TGACCCCATC |
| 17041 | TCATTGGGTC CCAGCCCTGC ACAGACATTT TTTTAGTCTT CCTCCGGTTG AATCCTATAA |
| 17101 | CCACATTCTT GCCTCAGTGT ATCCACAGAA CATCCAAACC CAGGGACGAG TGTGGATACT |
| 17161 | TCTTTGCCAT TCTCCGCAAC TCCCAGCCCA GAGCTGGAGG GTCTCAAGGA GGGGCCTAAT |
| 17221 | AATTGTGTAA TACTGAATAC AGCCAGAGTT TCAGGTCATA TACTCAGCCC TGCCATGCAC |
| 17281 | CGGCAGGTCC TAGGTGACCC CCGTCAAACT CAGTTTCCTT ATATATAAAA TGGGGTAAGG |
| 17341 | GGGCCGGGCG CAGTGGCTCA CGAATCCCAC ACTCTGGGAG GCCAAGGCGA GTGGATCACC |
| 17401 | TGAGGTCGGG AGTTTGAGCC CAGCCTGACC AACATGGAGA AACCCCATCT CTACTAAAAA |
| 17461 | TACAAAAGTA GCCGGGCGTG GTGATGCATG CCTGTAATCC CAGCTACCTA CTCGGGAGGC |
| 17521 | TGAGGCAGGA GAATCGCTTG AACCCGGGAG GCAGAGGTTG CGGTGAGCTG AGATCTCACC |
| 17581 | ATTACACTCC AGCCTGGGCA ACAAGAGTGA AACTCCGTCT CAAAAAAGAT AAATAAAGTA |
| 17641 | AAATGGGGTA AGGGAAGATT ACGAGACTAA TACACACTAA TACTCTGAGG TGCTCAGTAA |
| 17701 | ACATATTTGC ATGGGGTGTG GCCACCATCT TGATTTGAAT TCCCGTTGTC CCAGCCTTAG |
| 17761 | GCCCTTCAAA GCATTGGTCA GGGAAAAGGG GCACAGACCC TCTCACTCAT GTGATGTCAT |
| 17821 | CTCTCCTCCC TGCTTCTGTC TCCTACAGCC ACCTGAAGTC CAAAAAGGGT CAGTCTACCT |
| 17881 | CCCGCCATAA AAAACTCATG TTCAAGACAG AAGGGCCTGA CTCAGACTGA CATTCTCCAC |
| 17941 | TTCTTGTTCC CCACTGACAG CCTCCCACCC CCATCTCTCC CTCCCCTGCC ATTTTGGGTT |
| 18001 | TTGGGTCTTT GAACCCTTGC TTGCAATAGG TGTGCGTCAG AAGCACCCAG GACTTCCATT |
| 18061 | TGCTTTGTCC CGGGGCTCCA CTGAACAAGT TGGCCTGCAC TGGTGTTTTG TTGTGGGGAG |
| 18121 | GAGGATGGGG AGTAGGACAT ACCAGCTTAG ATTTTAAGGT TTTTACTGTG AGGGATGTTT |
| 18181 | GGGAGATGTA AGAAATGTTC TTGCAGTTAA GGGTTAGTTT ACAATCAGCC ACATTCTAGG |
| 18241 | TAGGGGCCCA CTTCACCGTA CTAACCAGGG AAGCTGTCCC TCACTGTTGA ATTTTCTCTA |
| 18301 | ACTTCAAGGC CCATATCTGT GAAATGCTGG CATTTGCACC TACCTCACAG AGTGCATTGT |
| 18361 | GAGGGTTAAT GAAATAATGT ACATCTGGCC TTGAAACCAC CTTTTATTAC ATGGGGTCTA |
| 18421 | GAACTTGACC CCCTTGAGGG TGCTTGTTCC CTCTCCCTGT TGGTCGGTGG GTTGGTAGTT |
| 18481 | TCTACAGTTG GGCAGCTGGT TAGGTAGAGG GAGTTGTCAA GTCTCTGCTG GCCCAGCCAA |
| 18541 | ACCCTGTCTG ACAACCTCTT GGTGAACCTT AGTACCTAAA AGGAAATCTC ACCCCATCCC |
| 18601 | ACACCCTGGA GGATTTCATC TCTTGTATAT GATGATCTGG ATCCACCAAG ACTTGTTTTA |
| 18661 | TGCTCAGGGT CAATTTCTTT TTTCTTTTTT TTTTTTTTTT TTCTTTTTCT TTGAGACTGG |
| 18721 | GTCTCGCTTT GTTGCCCAGG CTGGAGTGGA GTGGCGTGAT CTTGGCTTAC TGCAGCCTTT |
| 18781 | GCCTCCCCGG CTCGAGCAGT CCTGCCTCAG CCTCCGGAGT AGCTGGGACC ACAGGTTCAT |
| 18841 | GCCACCATGG CCAGCCAACT TTTGCATGTT TTGTAGAGAT GGGGTCTCAC AGTGTTGCCC |
| 18901 | AGGCTGGTCT CAAACTCCTG GGCTCAGGCG ATCCACCTGT CTCAGCCTCC CAGAGTGCTG |
| 18961 | GGATTACAAT TGTGAGCCAC CACGTCCAGC TGGAAGGGTC AACATCTTTT ACATTCTGCA |
| 19021 | AGCACATCTG CATTTTCACC CCACCCTTCC CCTCCTTCTC CCTTTTTATA TCCCATTTTT |
| 19081 | ATATCGATCT CTTATTTTAC AATAAAACTT TGCTGCCACC TGTGTGTCTG AGGGGTGAAC |
| 19141 | GCCAGTGCAG GCTACTGGGG TCAGCAGGTG CAGGGGTGAG TGAGGAGGTG CTGGGAAGCA |
| 19201 | GCCACCTGAG TCTGCAATGA GTGTGGGCTG GGGGGCCCAG TGCCCGGGTT CCGGGAGGGG |
| 19261 | AACAAAGGCT GGAGACTGGG TCAGTCTGCG GGCTGCATGA CAACAAGGGA GGGGGTGGCT |
| 19321 | CCATTCATAA CTCAGGAACC AACCGTCCCT CCTCCCCTCC GGCCACGGCT GGCACAAGGT |
| 19381 | TCTCTCCCTC CCCTGCTTCT AGGACTGGGC TGCTTCCCCC TCGGCAGCCT CTCACCAAGG |
| 19441 | ATTACGGGAT TTAAATGTCT GATTTAGCAA GGCTGAGCCT CCAGGGTGGC CATCTGCTCC |
| 19501 | ATCAGAAAGT GGCAGGATAC CTGGGTTCCC AAGGGGAACA GGGGTGGGTG CTACTGGATG |
| 19561 | GAGAGAGGCC AGTGGGAGGC CTGCTAGCCA GGGTCCCAGG AAAGTGGGGG CAGCTAAGGT |
| 19621 | AAGAGTAGGG GTGTGGGGCT AGGTCCTTCC CAGCATCCCC TCATCCTGGG CCTCATGCCA |
| 19681 | GGTAGCTGAA TGAATTGAAG CTTTAAACTC TGCCAGGAAA ACCTTTCAAA GGGCTTCTTG |
| 19741 | GGATAGGGAG GAGAGTCGGG TTGAGGAGCT CAGTACTGCC TGCCCATGCT CCTCAGGGCT |
| 19801 | GCTGGCTCCC AGGGAGGGGG GCTGGGAGCA GGCAGGCTCT TCCCCATCAC CCACTGCTCT |
| 19861 | CTTGGAGCCA GTGCTTGAAG GGGCAGTCAG ACATGGCTTG CCCTTCCTCC TCCCTGGTGG |
| 19921 | TGGAGATGGG TGTTAGGGTC CAGTGGGTGC TACTGTCCAG GGGGGCTTCT GGGGCCACCA |
| 19981 | GCCTGTCAGC TCATCAACCA GGCTGAAGGT GCAAGCAGGA GCCCCTTGCC TTGCCCCAAG |
| 20041 | GATCCCAGAC AGCTATGAAG CCACCAGCCT TCCTGACCTC AAGACCACCT TTTTTTTTTC |
| 20101 | TCTTTCTTAC TAGGGAATGC CAAACACTCT CCCCAGGAGA TCCAGACCCG CCTCTTTCAG |
| 20161 | AGACTTTTAA CTTAAACATC TGTCCCTACC CAGCAGGCAA ACTAGAGCTC CTGAAGCTCA |
| 20221 | GTCCCTGTCC TTGCCTCTGT AGACAGGTCA CCTTGATGAG CTTCCTTTTT TTTTTTTTAA |
| 20281 | TTTTTTTTTA TTTTAGGCTT TATTGGGGCA TAATTGATCC CCCAAAATTG CATACATTCA |
| 20341 | AGGTATGCAG TGTGATGATT TGATATGGGG GTATATTGTG AAACCATTAC CACAATCAAA |
| 20401 | TTAATCAGCA CGTCCATCAT CACACACAGT TACCATTTGT GTGTGTGCAC GTGTGTTCAC |
| 20461 | CTACGACGAG GACACTTGGA CCTACTCTGC AGATCTCAAG TAAACAGAAA ATCTCCCTTT |
| 20521 | TTGACAACCA TCCTCCACCC TTTCAATCCC AACCTTTTCC TAGATTATGT CCCTAGCTCT |
| 20581 | GTTTTTATTT CTGCTGTGCT GCTTCAGATC CATTCTGACT CTGCCAAACC CTTCTTTGTG |
| 20641 | AGCTGATAGA TTGCTGGATT GAGAATTACA GCTGGGCGCG GTGGCTCACG CCTGTAATCC |
| 20701 | CAACACTGTG GGAGGCCAAG GCCGGCGGAT CACTTGAGGT CAGGAGTTGG AGACCAGCCT |
| 20761 | GACCAACAAG ATGAAACCCC ATCTCTACTA AAAATACAAA ATTAGCTGGG CATGGTGGTG |
| 20821 | CACGCCTGTA ATCTCATCTT CTTGGGAGGC TGAGGCAGGA GAATTGCTTG AACCCGGGAG |
| 20881 | GTGGAGGTTG CAGTGAGCCA AGATCCTGCC ATTGCACTCC AGCCTGGGCA ACAACAGTGA |
| 20941 | AGCTCCATCT CAAAACACAC AAAAAAAAGA AGTACAAAGT CTGAGACTTC AGGCCAGCTC |
| 21001 | TGCTACACTA TATACTCTAA CCTCTCTGGT CCTACTTGGT GACTTCTTTC CCTCTGGTCG |
| 21061 | TGTTCAAGTT CCCGTCCCAT CCAGTCAAGC AGGTACTCAT TGGTACCTTA CCCTGTGCCA |
| 21121 | GGAGCTGTTC TAGGCCCTGG AAACCTATGG CAGACATGTT CCCTACCCTC CCACTCAAAG |
| 21181 | AGCCCAGGCC TTATCCTAAT GAGATCTGAA ATCAAATCTC CCAATTTCCT CATGGCTTCA |
| 21241 | GTCTAAACTT GTAATTCACA ACCTTAAATC AATATGTTCT ATTTTTTTAT TTAGAAAACA |
| 21301 | TTTCCGGCCA GGCACGGTGG ATCACACCTG TAATCCCAGC TACTCGGGAG GCTGAGGCAG |
| 21361 | GAGAATCGCT TGAACCCAGG AGGCAGAGGG TTGCAGTGAG CCGAGATTGC GCCATTGCAC |
| 21421 | TCTAGCCTGG GCAACAGAGC AAGACTCCAT CTCAAAAAAG AAAAAAAAAT GGAAGAAAAA |
| 21481 | AAAATTTCCC CCTCATTTTA GGAACACGAG GTCTCCAAAT CTAAAATTCG TACTCTGAGG |
| 21541 | AGATTGAATA GCCTTAAATG CTTTCATCAT TAAAAAGAAA AGAAAGGAAC CTGGTATGCA |
| 21601 | TCCTAAAAAT GAAAAATATA CCTACCTGTA ATCCCAGCAC ACAGCACATT GGGAGGCTAA |
| 21661 | AGCAGGAGGA TAACTTGAGG CCAGGAGTTT CAGATCAGCC TGGGCAACAT AGCAACACCC |
| 21721 | CATTTCTTTT TCTTTTCTTT TTTTTTTGGA GACACAGTCT CGCTCTGTTA CTCAGGCTGG |
| 21781 | AGTGCAGTGG CTCAATCTCA GCTCACTGCA AGCTCTGCCT CCCAGGTTCA TGCCATTCTC |
| 21841 | CTGCCTCAGC CTCCCGAGTA GCTGGGACTA CAGGCGCCCG CCACCACGCC TGGCTAATTT |
| 21901 | TTTGTATTTT TAGTAGAGAC AGGGTTTCAC CGTGTTAGCC AGGATGGTCT CGATCTCCTG |
| 21961 | ACCTCGTGAT CCGCCAGCCT TGGCCTCCTA AAGTACTGGG ATTACAGGCG TGAGCCACTG |
| 22021 | CGCCTGGCCA CAACACCCCA TTTCTATTTT AATAAAATAA AATACTGTGA AAAACATTTA |
| 22081 | CAATTTTTAA ATTTTAATTT TAAAATTAAA CTTATATTTA TTCATTTGTG TGTGTGGGTT |
| 22141 | TTTTTTTTTT TTTTTTTTTG CTTTTTTTTT GAGATGGAGT GTCACTCTGT CACCCAGGCT |
| 22201 | GGAGTGCAGT GGCGTGATCT CTGCCTCCCG GTTCAAGTGA TTCTCCTGCC ATAGCCTCCC |
| 22261 | AAGTAGCTGG GACTACAGGT ACACGCCACC ACGCCGGGTT AATTTTTGTA TTTTTAGTAG |
| 22321 | AGACAGGATT TCACTGTGTC GCCAGGCTAG CCTCGAACTC CTGACCTCAG GTGATTCGCC |
| 22381 | CACCTTGGCC TCCCAAAGTG CTGTGATTAC AAGCGTGAGC CACCGTGCCC AGCCCAAAGT |
| 22441 | TGGTTTTAAT AGCAGAAAAT CTATCAACAT AATTCAATAT ATTAAATTTA GAAAGAAAAA |
| 22501 | TTATCTATCA TATCAACAGA TACTGAAAGG AATTTGATTA AATTTCAGTA GCCATTTCCT |
| 22561 | TAAAAAAGAA AACACTTTAA CACAGTAATA GACTGATAAT GGAATACCAA TTTTCCTAAT |
| 22621 | AAGTTAAACA TTAAGATAAT TTCAATTAAG GTCAAGAGCT GGGCCAGGTG CAGTGGCTCA |
| 22681 | CACCTGTAAT CCCAACACTT TGGAGGCCAA GGTGGGTGGA TCACCTGAGG TCAGGAGTGG |
| 22741 | AGACCAGCCT GGCTGACAAT AGTGAAATCC TGCCTCTACT AAAAACACAA AAAATTAGCT |
| 22801 | GGGCATGGTG GTGGGCACCT ATAATCCCAG CTACTGGGAA GGCTGAGACA GGAGAATTGC |
| 22861 | TTGAACCTGG GAGGCGGAGG TTGCAGTGAG CAAAGATCAC ACCATTGCAC TCCAGCCTGG |
| 22921 | GCGACAGAGC CAGAGTCAGT CTCAAAAAAA AAAAGAGGTG GCCACACCTA TAATCCAAAC |
| 22981 | ATTTTGTGAG GCCAAGGCAG GAGAATTGCT TCAGGCCAAG AGTTGAACAC CTCGTCAACA |
| 23041 | TAGCCAGACC TCTCTCTAGA TAGATAGATA GATGATAGAT AGAGAGATAG ATAGATGATA |
| 23101 | GATAGAGAGA TAGATAGATG ATAGATAGAT AGATAGATAG ATAGATAGAT AGATAGATAG |
| 23161 | ATAGATAGAT AGATAGATAA TCTGGCCGGG TGTGGAGGCT CACGCCTGTA ATCCCAGCAC |
| 23221 | TTTGGGAGGC TGAGGCGGGC AGATCACGAG GACAAGAGAT TGAAACCATC CTGGCTAACA |
| 23281 | AGGTGAAACC CCGTCTCTAC TAAAAATACA AAAAATTAGG CGGGTGTGGT GGCACGCGCC |
| 23341 | TGTAGTCCTA GCTATTCAGG AGGCTGAGAC AGGAGAATTG CTTGAATCCG AAAGGCGGAG |
| 23401 | GTTGCAGCGA GCCGAGATCG TGCCACTGCA CTCCAGCCTG GGTGACAGAG CAAGACTCCA |
| 23461 | TCTCAAAATA AATAAATAAA TAATCAAGAA CAGTATAAGG GGCTGTATGG TGGCTCATGC |
| 23521 | CTGTGATCCC AGCACTTTGG GAGGCCAAGG TGGGAGGATC CCTTGAGACC AGCCCAGGCA |
| 23581 | ACAGAGAAAG ACCCTGTCTC TATTTAAAAA AATTAAAAAC TGGCCGGGCA CGGTGGCTCA |
| 23641 | CGCCTGTAAT TCCAGCGCTT GGGAGGCCAA GGCAGGCACA TCAGGAGGTC AGGAGTTCGA |
| 23701 | GACCAGCCTG GCCAACGTGG TGAAACCCCG TCTCTACTAA AAATACAAAA AGTAGCTAGG |
| 23761 | CGTGGTGGCA GGCACCTGTA ATCCCAGCTA CTTGGGAGGC TGAGGCAGGA GAATCGCTTG |
| 23821 | AACCCAGGAG GCGGAGGTTG CAGTGGGCAA AGATCGTGCC ATTGCACTCA GCCTGGGTGA |
| 23881 | CAGGGCAAGA CTCCATCTCA AAATAAATAA ACAAAGTAAT TAATTAATTA AATTAAAAAC |
| 23941 | TGTGGGGATA TAGACTTACT CTGGTTTTAT TTTTTCTTTT CTTTTCTTTT CTTTTTTCTG |
| 24001 | AGACGGAGTC TCGCTCTGTT GCCCAGGCTG GAGTACAGTG GCGTGGTTTC TGTTCTCTGC |
| 24061 | AACCTCCACC TCCCGGATTC AAGCGATTCT CTTGCCTCAG CCTCTTGAAT ACCTGGAATT |
| 24121 | ACAGGTGCCT GCCACCACCC CCGGCTAATT TTTTGTATTT TTAGTAGAGA CAGGGTTTCA |
| 24181 | CCATGTTGGC CAAGCTGGTC TCGAACTCCT GACCTCATGA TCCACCCGCC TCTGCCTCCC |
| 24241 | AAAGCACTGA GACTACAGGA GTGAGCCACT GTGCCCAGCC TACTCTGGTT TTAGTGCATT |
| 24301 | CAAGAGGAAC AAAAAAGGAA GAAAATCACT AGTAAATATA CCTCTTTCTG GTTAGAGTGG |
| 24361 | ATGTTTGGAA ATTATATATA TATTATATTA TATTATATAT ATTATATATA TACACAAACA |
| 24421 | CGTACATACA TGCACACACA TATATGCCTT TTTGATTATA GGATAGTATA CCAAAACTCA |
| 24481 | GAAATATTAT GGAATTAACA GAATTTAGTA AGGCAGATAA GTAGTAGGTA GAAAAATATT |
| 24541 | AATTTTATCT TCCAGCAGAA GCACTGTGAA AAATTAGACA ACAAGAAAAC ATTCCATTCA |
| 24601 | AAATAATGAC AATAAGGCCG GGCATGGTGG CTCACACCTG TAATCCCAGC ACTTTGGGAG |
| 24661 | GCTGAGGCAG GAGGATCATC TGAGGTCAAG TTTGAGATCA GCCTGGCCAA CATGGTGACA |
| 24721 | CCCTGTCTCT ACTGAAAATA CAAAAATCAG CCAGCTATGG TAGTGTAAGC CTGTAATTCC |
| 24781 | AGCTACTCGG GAGGTCGAAG CAGAAGAATC ACTTGAACCC AGGAGGCAGA GATTGCAGTG |
| 24841 | AGCCAAGATC CTGCCAGTGC TTTCCAGCCT GGGCAACAGT GTGAGGCTCC ATCTCAAAAA |
| 24901 | AAAAAAAAAA AAAAAGACAA TAGCAATAAA CATTAAGAAA TGTGTAATAG GAATGGCACA |
| 24961 | CACAAAGAAG GAATGGCACA GAGCCTGTAT GCAGAAGACC ACAAACCCTT ATTTAACGAC |
| 25021 | GTAAGCCAAG ATCCAAAGAA AATGATAGAT TCTCAGATGG GAAAACTAAA AAAATAAGAA |
| 25081 | AAATCAATTA TCTCGAGATA AATATAATAT AATGCAATTT CAATTAGAAT CCCAAATTTT |
| 25141 | CATTGTGTGT GTGTGTGAGT TGGGTAAATT TATCATAAAT GTATAGGAAC GAGTAAGTGT |
| 25201 | CACTAGTTGT TTAAATAAAT ACTGGATTTG GGCCAGGCAT GGTGGCTCAC GCCTCTAATC |
| 25261 | CCAGCACTTT GGGAGACCGA GGCGGGCAGA TCATGAGGTC AGGAGATCGA GACCATCTGG |
| 25321 | CCAACATAGT GAAAACTCGT CTCTACTAAA GATACAAAAA ATTAGCTGGG CATGGTGGCA |
| 25381 | CGTGCCTGTA GTTCCAGCTA CTCTGGAGGC TGAGGCAGGA GAGTTGCTTG AACCCGGGAG |
| 25441 | GTGGAGGTTG CAATGAGCCG AGATCCTGTC ACTGCACTCC ACCCTGGCGA CAAAGTGAGA |
| 25501 | CTCCGTCTCT CTCTCTCTCT TTAGGCCAAG GCAGGTGGAT CACCTGAGGT CAGGAGTTCA |
| 25561 | AGACAGCCTG GCCAACATAG CGAAATCCCA TCTCTACTAA AAATACAAAA ATTAGCCTGG |
| 25621 | CAGTGGTGGC CCACGCCTGT AATCCCAGCT ACTAAGGGGG CTGAGGCAGG AGGATCTCTT |
| 25681 | AACCAGGGAG GAGGAGGTTG CAGTGAGCAG AGATTGTGCC ACTGCACTCC AGCCTGTGCA |
| 25741 | ACAGAGTGAG ACTCTGTCTC |

### Coding Regions

Exon 1 includes Seq. ID No. 1 base numbers: 1-162.

Exon 2 includes Seq. ID No. 1, base numbers: 10917-11018.

Exon 3 includes Seq. ID No. 1, base numbers: 11136-11157.

Exon 4 includes Seq. ID No. 1, base numbers: 11267-11545.

Exon 5 includes Seq. ID No. 1, base numbers: 12303-12486.

Exon 6 includes Seq. ID No. 1, base numbers: 12568-12680.

Exon 7 includes Seq. ID No. 1, base numbers: 13249-13358.

Exon 8 includes Seq. ID No. 1, base numbers: 13702-13838.

Exon 9 includes Seq. ID No. 1, base numbers: 13931-14004.

Exon 10 includes Seq. ID No. 1, base numbers: 16824-16930.

Exon 11 includes Seq. ID No. 1, base numbers: 17849-19137.

Table 1 includes the nucleotide sequences for Exons 1-11, which uses the base numbers with reference to Seq. ID No. 1 to determine the individual sequences.

TABLE 1
Exon nucleotide sequences.

| Exon | |
|---|---|
| ID No. | Sequence |
| 1 | GTTTTCCCCT CCCATGTGCT CAAGACTGGC GCTAAAAGTT TTGAGCTTCT CAAAAGTCTA |
| | GAGCCACCGT CCAGGGAGCA GGTAGCTGCT GGGCTCCGGG GACACTTTGC |
| | GTTCGGGCTG GGAGCGTGCT TTCCACGACG GTGACACGCT TCCCTGGATT GG |
| 2 | CAGC CAGACTGCCT TCCGGGTCAC TGCCATGGAG GAGCCGCAGT CAGATCCTAG |
| | CGTCGAGCCC CCTCTGAGTC AGGAAACATT TTCAGACCTA TGGAAACT |
| 3 | ACTTC CTGAAAACAA CGTTCTG |
| 4 | TCCC CCTTGCCGTC CCAAGCAATG GATGATTTGA TGCTGTCCCC GGACGATATT |
| | GAACAATGGT TCACTGAAGA CCCAGGTCCA GATGAAGCTC CCAGAATGCC |
| | AGAGGCTGCT CCCCCCGTGG CCCCTGCACC AGCAGCTCCT ACACCGGCGG |
| | CCCCTGCACC AGCCCCCTCC TGGCCCCTGT CATCTTCTGT CCCTTCCCAG AAAACCTACC |
| | AGGGCAGCTA CGGTTTCCGT CTGGGCTTCT TGCATTCTGG GACAGCCAAG |
| | TCTGTGACTT GCACG |
| 5 | TACTCCCC TGCCCTCAAC AAGATGTTTT GCCAACTGGC CAAGACCTGC CCTGTGCAGC |
| | TGTGGGTTGA TTCCACACCC CCGCCCGGCA CCCGCGTCCG CGCCATGGCC |
| | ATCTACAAGC AGTCACAGCA CATGACGGAG GTTGTGAGGC GCTGCCCCCA |
| | CCATGAGCGC TGCTCAGATA GCGATG |
| 6 | GTC TGGCCCCTCC TCAGCATCTT ATCCGAGTGG AAGGAAATTT GCGTGTGGAG |
| | TATTTGGATG ACAGAAACAC TTTTCGACAT AGTGTGGTGG TGCCCTATGA |
| | GCCGCCTGAG |
| 7 | GT TGGCTCTGAC TGTACCACCA TCCACTACAA CTACATGTGT AACAGTTCCT |
| | GCATGGGCGG CATGAACCGG AGGCCCATCC TCACCATCAT CACACTGGAA GACTCCAG |
| 8 | TGGTAATCT ACTGGGACGG AACAGCTTTG AGGTGCGTGT TTGTGCCTGT CCTGGGAGAG |
| | ACCGGCGCAC AGAGGAAGAG AATCTCCGCA AGAAAGGGGA GCCTCACCAC |
| | GAGCTGCCCC CAGGGAGCAC TAAGCGAG |
| 9 | CACTGCCCAA CAACACCAGC TCCTCTCCCC AGCCAAAGAA GAAACCACTG |
| | GATGGAGAAT ATTTCACCCT TCAG |
| 10 | ATCCGTG GGCGTGAGCG CTTCGAGATG TTCCGAGAGC TGAATGAGGC CTTGGAACTC |
| | AAGGATGCCC AGGCTGGGAA GGAGCCAGGG GGGAGCAGGG CTCACTCCAG |
| 11 | CC ACCTGAAGTC CAAAAAGGGT CAGTCTACCT CCCGCCATAA AAAACTCATG |
| | TTCAAGACAG AAGGGCCTGA CTCAGACTGA CATTCTCCAC TTCTTGTTCC |
| | CCACTGACAG CCTCCCACCC CCATCTCTCC CTCCCCTGCC ATTTTGGGTT TTGGGTCTTT |
| | GAACCCTTGC TTGCAATAGG TGTGCGTCAG AAGCACCCAG GACTTCCATT |
| | TGCTTTGTCC CGGGGCTCCA CTGAACAAGT TGGCCTGCAC TGGTGTTTTG |
| | TTGTGGGGAG GAGGATGGGG AGTAGGACAT ACCAGCTTAG ATTTTAAGGT |
| | TTTTACTGTG AGGGATGTTT GGGAGATGTA AGAAATGTTC TTGCAGTTAA |
| | GGGTTAGTTT ACAATCAGCC ACATTCTAGG TAGGGGCCCA CTTCACCGTA |
| | CTAACCAGGG AAGCTGTCCC TCACTGTTGA ATTTTCTCTA ACTTCAAGGC CCATATCTGT |
| | GAAATGCTGG CATTTGCACC TACCTCACAG AGTGCATTGT GAGGGTTAAT |
| | GAAATAATGT ACATCTGGCC TTGAAACCAC CTTTTATTAC ATGGGGTCTA |
| | GAACTTGACC CCCTTGAGGG TGCTTGTTCC CTCTCCCTGT TGGTCGGTGG GTTGGTAGTT |
| | TCTACAGTTG GGCAGCTGGT TAGGTAGAGG GAGTTGTCAA GTCTCTGCTG |
| | GCCCAGCCAA ACCCTGTCTG ACAACCTCTT GGTGAACCTT AGTACCTAAA |
| | AGGAAATCTC ACCCCATCCC ACACCCTGGA GGATTTCATC TCTTGTATAT |
| | GATGATCTGG ATCCACCAAG ACTTGTTTTA TGCTCAGGGT CAATTTCTTT TTTCTTTTTT |
| | TTTTTTTTTT TTCTTTTTCT TTGAGACTGG GTCTCGCTTT GTTGCCCAGG CTGGAGTGGA |
| | GTGGCGTGAT CTTGGCTTAC TGCAGCCTTT GCCTCCCCGG CTCGAGCAGT CCTGCCTCAG |
| | CCTCCGGAGT AGCTGGGACC ACAGGTTCAT GCCACCATGG CCAGCCAACT |
| | TTTGCATGTT TTGTAGAGAT GGGGTCTCAC AGTGTTGCCC AGGCTGGTCT CAAACTCCTG |
| | GGCTCAGGCG ATCCACCTGT CTCAGCCTCC CAGAGTGCTG GGATTACAAT |
| | TGTGAGCCAC CACGTCCAGC TGGAAGGGTC AACATCTTTT ACATTCTGCA |
| | AGCACATCTG CATTTTCACC CCACCCTTCC CCTCCTTCTC CCTTTTTATA TCCCATTTTT |
| | ATATCGATCT CTTATTTTAC AATAAAACTT TGCTGCCACC TGTGTGTCTG AGGGGTG |

TP53 Knock Out in MCF7 Cells with CRISPR Cas9

TP53 knockout MCF7 cells were generated as previously published [1]. Human codon-optimized Streptococcus pyogenes wild type Cas9 (Cas9-2A-GFP) was obtained by Addgene (Cat. No. 44719). Chimeric guide RNA expression cassettes with different sgRNAs (sgRNA1: CCATTGTTCAATATCGTCCG; sgRNA2: GACGGAAACCGTAGCTGCCC; sgRNA3: TGGTTATAGGATTCAACCGG) were ordered as gBlocks. These gBlocks were amplified by PCR using primers: gBlock_Amplifying_F: 5′-GTACAAAAAAGCAGGCTTTAAAGG-3′ and gBlock_Amplifying R: 5′-TAATGCCAACTTTGTACAAGAAAGC-3′. The PCR product was purified by Agencourt Ampure XP PCR Purification beads per the manufacturer's protocol (Beckman Coulter). One microgram of Cas9 plasmid and 0.3 μg of each gRNA gBlock (pair 1: sgRNA1 & sgRNA2; pair 2: sgRNA1 & sgRNA3) were cotransfected into MCF7 cells via Lipofectamine 3000 in a 6-well plate. Knockout cells created using the pair sgRNA1 & sgRNA2 were named KO5.6, and knockout cells created using the pair sgRNA1 & sgnRNA3 were named KO3.4. Knockout pools were cultured in 10 μM Nutlin-3a (SelleckChem) for 2 months, changing nutlin-3a treated media every 3 days and passaging cells every 6 to 8 days. Isogenic clones were isolated from the knockout and wild type pools via limiting dilution in a 96-well plate and incubated at 37° C. in a CO2 incubator for 15 days. Wild type clones were named Parental (PR).

### Sanger Sequencing

DNA was isolated from each MCF7 wild type pool, knockout pool, and single cell clone following the Agencourt DNAdvance genomic DNA isolation kit. MCF7 wild type and KO5.6 cells were PCR amplified using primers: TP53_exon_4_F: 5′-CTGGTAAGGACAAGGGTTGG-3′ and TP53_exon_4_R: 5′-GCCAAAGGGTGAAGAGGAAT-3′. MCF7 KO3.4 cells were PCR amplified using primers: TP53_exon_4_F and TP53_Woke_R: 5′-ATTAGGCCCCTCCTTGAGAC-3′. Products were sent to Eton Bioscience Inc. who purified the PCR products and performed Sanger Sequencing.

### Western Blotting

Protein was extracted from cells using 2-Mercaptoethanol and Laemmli sample buffer (Bio Rad, Cat. No. 1610737). MCF7 wild type and knockout derivative proteins (10 μg) were separated on 4-12% gradient polyacrylamide gels via SDS-PAGE and transferred to PVDF membranes (Millipore). Primary antibody dilutions were 1:300 for TP53 and 1:500 for β-actin. Drug Screening

MCF7 wild type and knockout derivative pools were plated at a density of 5000 cells/well in polystyrene, flat-bottom 96-well plates. All 133 compounds from the NCI Approved oncology drugs set IV (Table 2, AOD-IV Drug) were dissolved in DMF or DMSO at 10 mM stocks. Cells were treated at concentrations from 156.25 nM to 10 μM for 10 days. DMSO or DMF vehicle was used as a negative control. After 10 days, resazurin (Sigma, Cat. No. R7017) was added to each well and incubated at 37° C. in a dark CO2 incubator for 4 to 6 hours. A microplate reader took optical measurements (ex: 535 nm/em: 585 nm). Drugs showing larger Area under the curve (AUC) differences based on fluorescence values between MCF7 TP53 wild type and knockout cell lines were selected and further characterized, notably: oxaliplatin, 5-fluorouracil, and palbociclib.

### Results

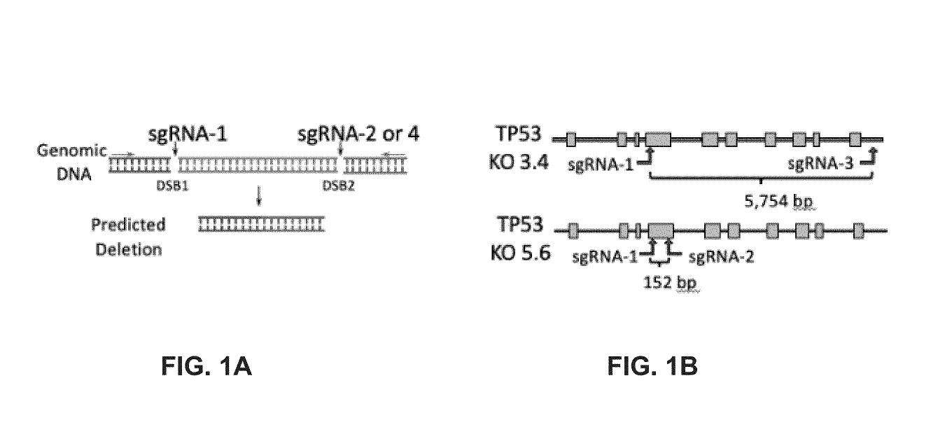

FIG. 1A illustrates an example knockout strategy/design and predicted deletion. FIG. 1B shows example targets of sgRNA1, 2, and 3 within TP53 of KO3.4 and KO5.6. sgRNA1 targets within exon 4, sgRNA2 targets downstream of sgRNA1 within exon 4, and sgNRA3 targets the intron after exon 10.

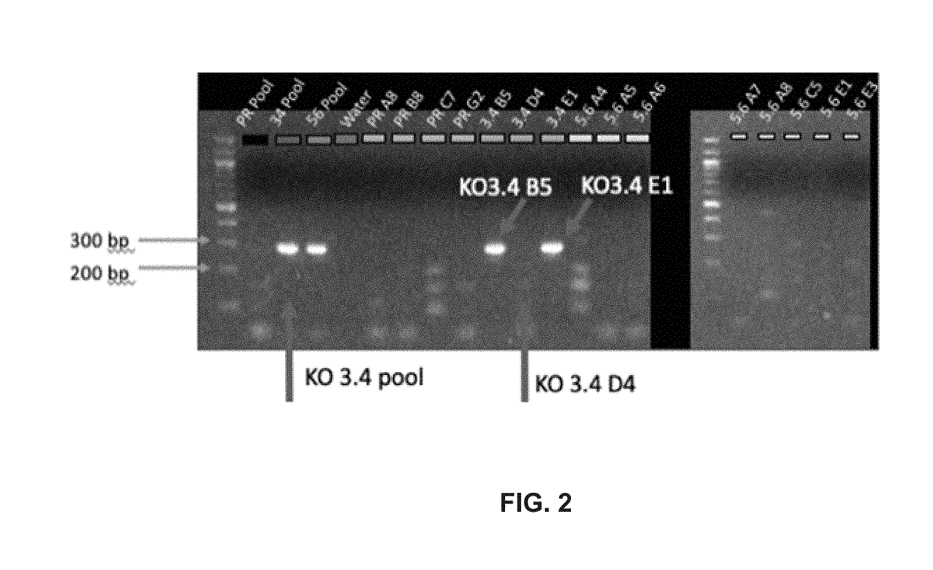

FIG. 2 illustrates an example gel shown using primers TP53_exon_4_F and TP53_Woke_R, only KO3.4 single cell clones with the predicted deletion should make a product. Single cell clones KO3.4 B5 and E1 made a product and wild type or KO5.6 cells did not, as expected.

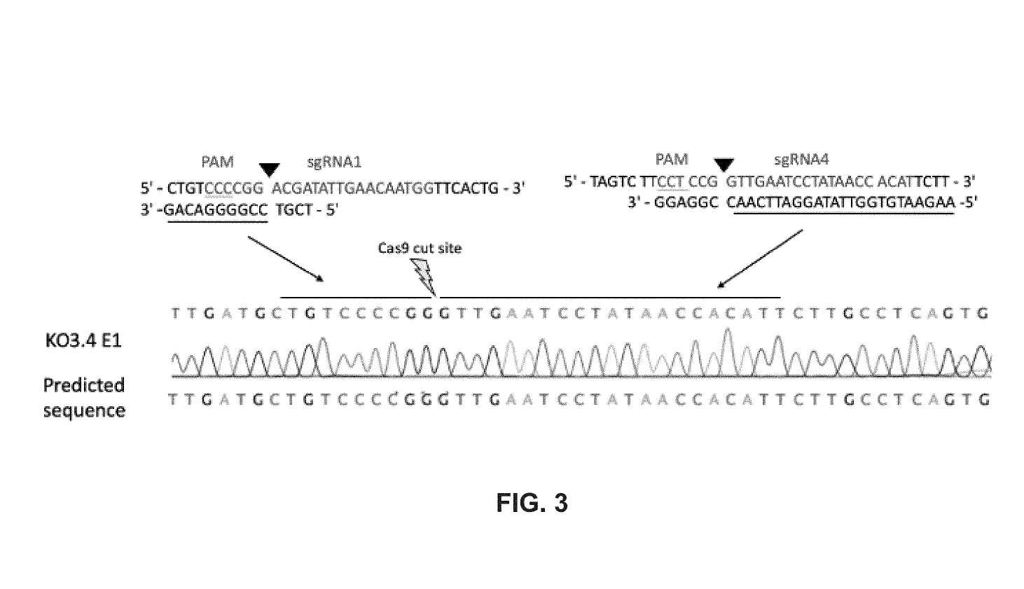

FIG. 3 illustrates an example Sanger sequencing of PCR product shows KO3.4 pool, KO3.4 B5, and KO3.4 E1 have the same sequence at the Cas9 cut site.

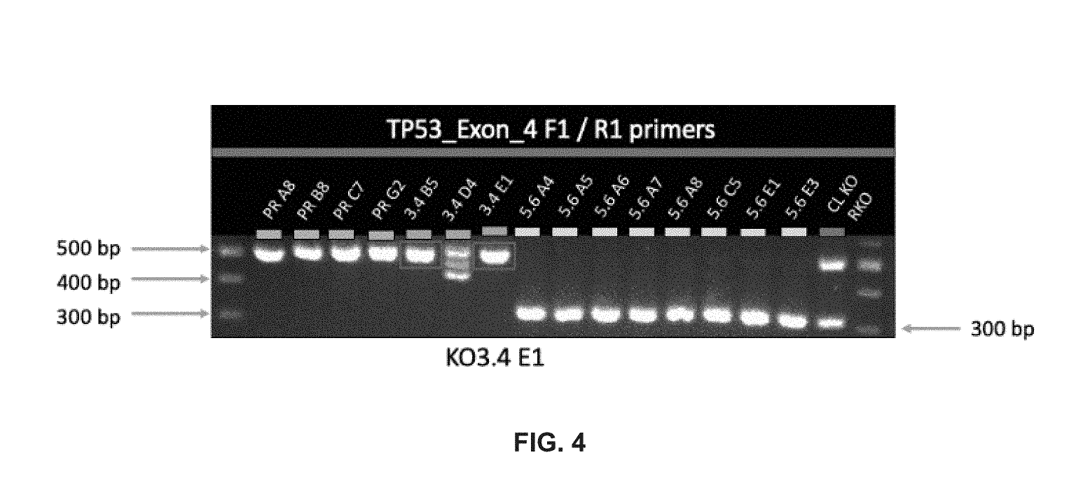

FIG. 4 illustrates an example gel shown using primers TP53_exon_4_F and TP53_exon_4_R, wild type exons should make a 496 bp product and TP53 KO5.6 knockouts should make a 344 bp product. KO3.4 B5 and E1 also display a wild type-sized product, indicating these knockouts have a large deletion on one allele and a wild type sized allele.

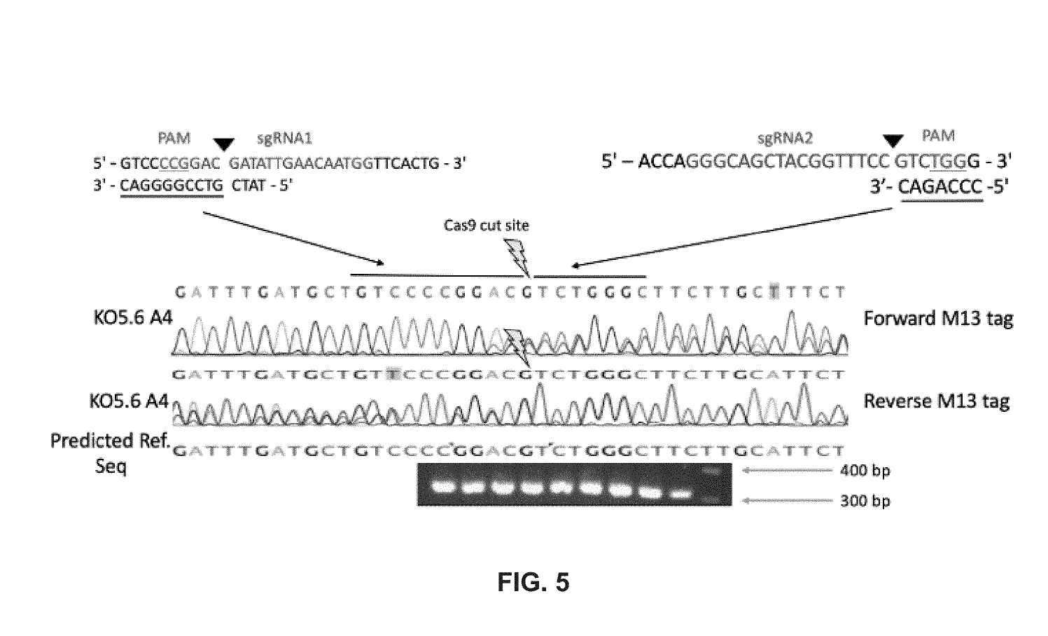

FIG. 5 illustrates an example Sanger sequencing of PCR product shows KO5.6 A4, A5, A6, A7, A8, and E3 at the Cas9 cut site. The KO5.6 sequence matches to the predicted deletion, but the sequence becomes rougher after the Cas9 cut site when reading from F or R. This can indicate that the two alleles were cut/repaired differently but the knockout was successful.

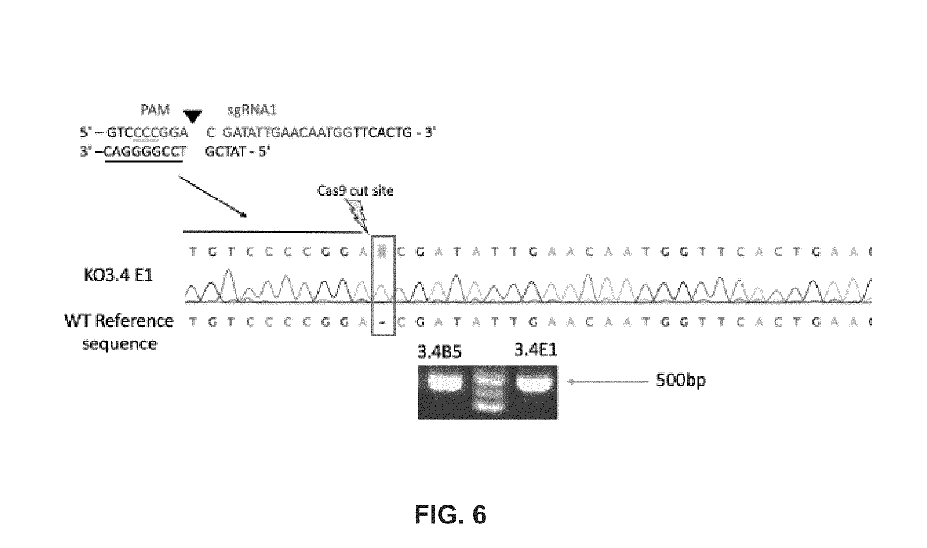

FIG. 6 illustrates an example knockout, KO3.4 E1, which shows an insertion of an “A” at the Cas9 cut site. The sequence is identical to wild type sequence otherwise. This frameshift causes the resulting protein to be non-functional and not p53.

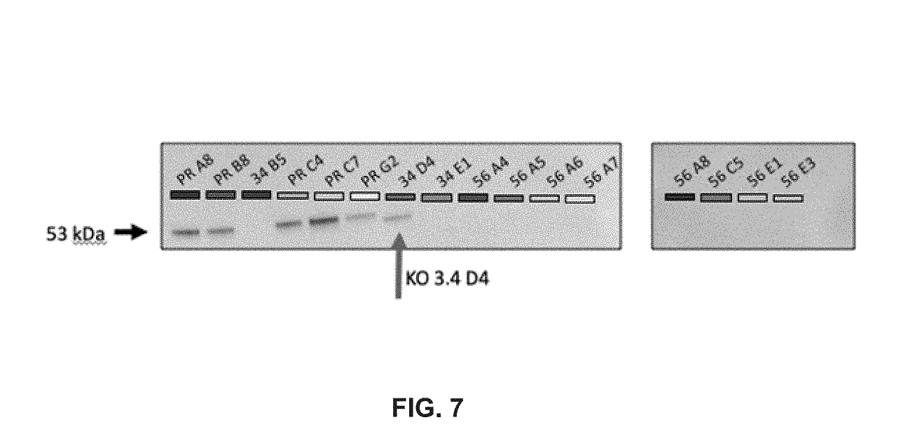

FIG. 7 illustrates an example western blot displaying protein in wild type cells, lesser or no protein in KO3.4 B5 and E1, KO5.6 A4, A5, A6, A7, A8, E1, and E3, and lesser or a truncated protein in KO3.4D4.

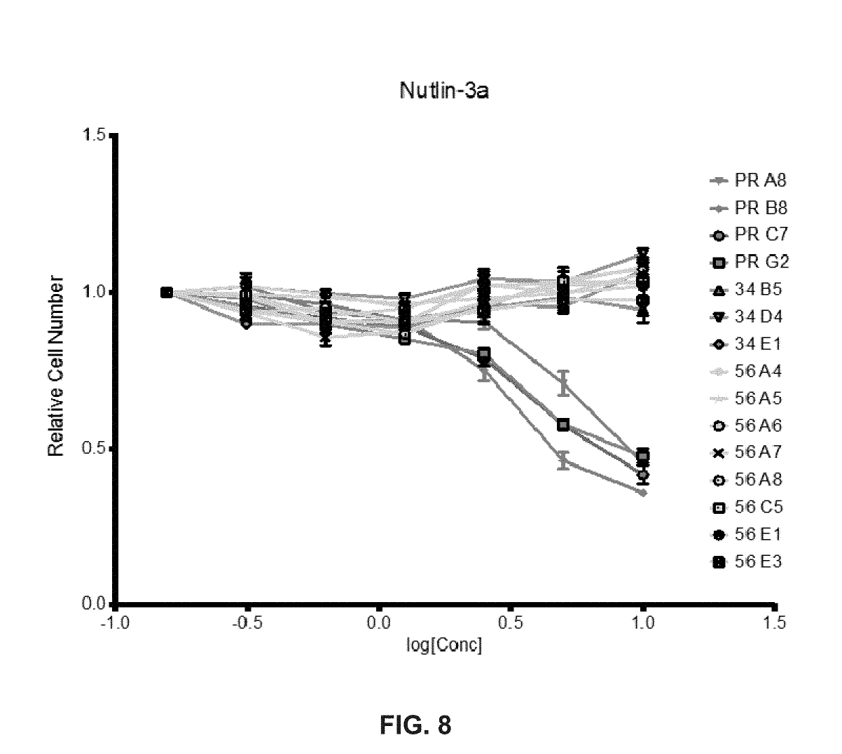

FIG. 8 illustrates an example plot displaying relative cell count versus concentration of Nutlin-3a. All knockout clones shown display some resistance to MDM2-inhibitor, Nutlin-3a. All wild type clones shown display some sensitivity. MDM2 binds to and degrades p53. Nutlin-3a competitively inhibits MDM2 and when functional p53 present, the cells undergo cell cycle arrest. Certain knockouts are resistant to nutlin up to 10 uM and wild types are more sensitive. Knockouts do not have a functional p53 protein.

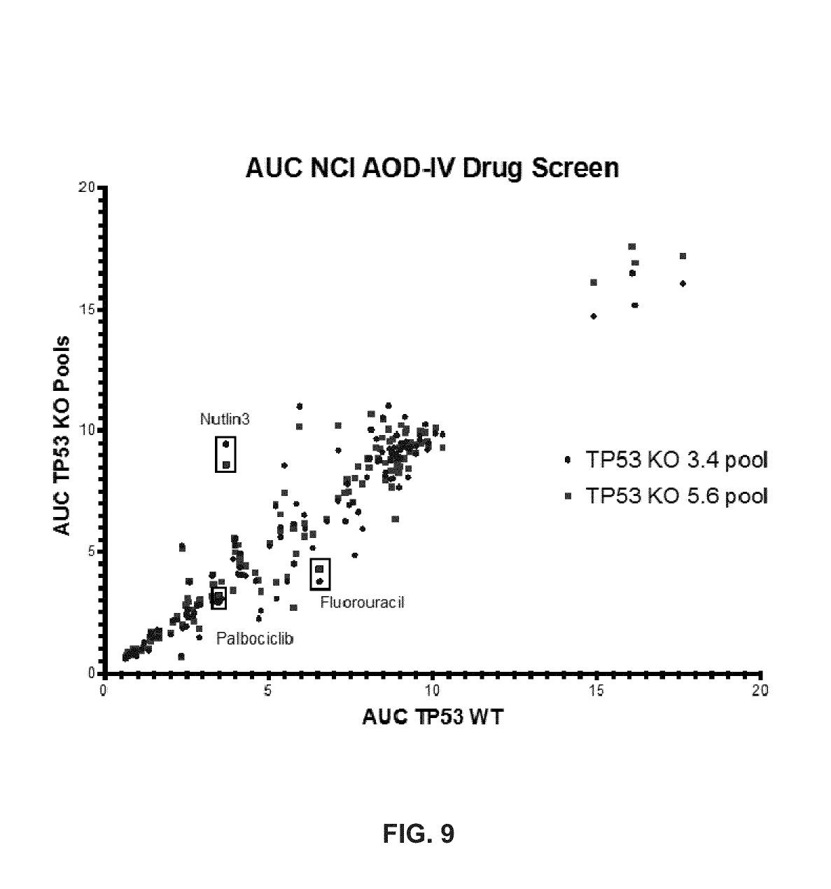

FIG. 9 illustrates an example plot displaying AUC of TP53 KO pools vs AUC of TP53 wild type pools following 10 day drug treatment. See Table 2 for data points.

TABLE 2
Approved oncology drug set IV Drugs and their respective
areas under the curve (AUC) upon ten-day drug treatment.

| | MCF7 WT | TP53 KO | TP53 KO |
|---|---|---|---|
| | Parental pool | 3.4 pool | 5.6 pool |
| AOD-IV Drug | (AUC) | (AUC) | (AUC) |
| Abiraterone | 8.378 | 8.785 | 9.254 |
| Afatanib | 2.734 | 2.48 | 2.139 |
| Alectinib | 5.763 | 4.51 | 2.7 |
| Allopurinol | 8.893 | 9.837 | 8.784 |
| Altretamine | 8.059 | 8.874 | 8.473 |
| Amifostine | 8.456 | 9.108 | 9.73 |
| Aminolevulinic Acid | 8.749 | 9.101 | 8.787 |
| Anastrozole | 8.01 | 8.067 | 8.828 |
| Arsenic Trioxide | 5.018 | 5.242 | 5.373 |
| Axitinib | 2.581 | 3.743 | 3.786 |
| Azactidine | 8.861 | 9.05 | 6.373 |
| Belinostat | 1.423 | 1.525 | 1.322 |
| Bendamustine Hydrochloride | 9.021 | 9.503 | 10.23 |
| Bleomycin Sulfate | 3.922 | 4.694 | 3.421 |
| Bortezomib | 0.6974 | 0.673 | 0.7486 |
| Bosutinib | 8.686 | 9.254 | 9.588 |
| Busulfan | 9.118 | 9.336 | 8.834 |
| Cabazitaxel | 1.406 | 1.658 | 1.713 |
| Cabozantinib | 4.126 | 4.376 | 4.52 |
| Capecitabine | 9.004 | 9.208 | 9.284 |
| Carboplatin | 8.83 | 9.302 | 8.343 |
| Carfilzomib | 0.6359 | 0.6462 | 0.6678 |
| Carmustine | 9.622 | 9.826 | 9.965 |
| Celecoxib | 8.779 | 9.053 | 9.654 |
| Ceritinib | 1.637 | 1.482 | 1.421 |
| Chlorambucil | 16.08 | 16.5 | 17.6 |
| Cisplatin | 7.335 | 6.27 | 7.44 |
| Cladribine | 5.569 | 3.767 | 3.956 |
| Clofarabine | 5.232 | 3.085 | 3.74 |
| Clyclopamine | 9.488 | 9.033 | 9.093 |
| Cobimetinib | 7.621 | 4.851 | 8.041 |
| Crizotinib | 2.549 | 2.325 | 2.941 |
| Cyclophosphamide | 16.16 | 15.2 | 16.92 |
| Cytarabine Hydrochloride | 4.771 | 2.567 | 3.381 |
| Dabrafenib Mesylate | 14.9 | 14.73 | 16.12 |
| Dacarbazine | 17.63 | 16.09 | 17.2 |
| Dactinomycin | 2.01 | 1.588 | 1.65 |
| Dasatinib | 4.282 | 4.014 | 4.43 |
| Daunorubicin Hydrochloride | 0.7798 | 0.8286 | 0.8964 |
| Decitabine | 4.065 | 4.095 | 5.295 |
| Dexrazoxane | 8.317 | 8.753 | 8.868 |
| DMSO | 9.021 | 9.503 | 10.23 |
| Docetaxel | 1.432 | 1.639 | 1.663 |
| Doxorubicin Hydrochloride | 0.9435 | 0.9013 | 1.009 |
| Enzalutamide | 8.931 | 8.864 | 8.634 |
| Epirubicin Hydrochloride | 0.8944 | 0.9399 | 1.019 |
| Erismodgib | 7.435 | 6.922 | 7.491 |
| Erlotinib Hydrochloride | 7.115 | 7.091 | 7.232 |
| Estramustine Phosphate Sodium | 9.864 | 9.491 | 9.348 |
| Etoposide | 2.93 | 2.818 | 3.06 |
| Everolimus | 4.699 | 2.251 | 3.834 |
| Exemestane | 8.564 | 8.275 | 8.69 |
| Floxuridine | 2.391 | 1.85 | 1.997 |
| FludarabinePhosphate | 8.551 | 8.135 | 7.936 |
| Fluorouracil | 6.548 | 3.782 | 4.286 |
| Fulvestrant | 4.6 | 3.797 | 4.145 |
| Gefintinib | 8.974 | 7.676 | 8.242 |
| Gemcitabine Hydrochloride | 2.888 | 1.464 | 1.827 |
| Hydroxyurea | 9.246 | 8.069 | 8.455 |
| Ibrutinib | 6.35 | 5.166 | 5.711 |
| Idarubicin Hydrochloride | 0.958 | 0.7035 | 0.8152 |
| Idelalisib | 7.566 | 7.039 | 7.03 |
| Ifosfamide | 9.772 | 10.26 | 9.935 |
| Imatinib | 8.269 | 9.674 | 9.312 |
| Imiquimod | 9.496 | 9.359 | 9.449 |
| Irinotecan Hydrochloride | 3.291 | 4.015 | 3.214 |
| Ixabepilone | 1.397 | 1.543 | 1.473 |
| Ixazomib | 0.719 | 0.7086 | 0.8766 |
| Laptinib | 5.364 | 5.619 | 5.857 |
| Lenalidomide | 8.724 | 8.782 | 8.801 |
| Letrozole | 9.288 | 9.219 | 9.099 |
| Lomustine | 9.62 | 9.629 | 9.491 |
| Mechlorethamine Hydrochloride | 6.077 | 6.543 | 6.171 |
| Megestrol Acetate | 9.824 | 9.221 | 9.555 |
| Melphalan Hydrochloride | 5.766 | 6.158 | 5.95 |
| Mercaptopurine | 6.103 | 5.941 | 5.636 |
| Methotrexate | 3.301 | 4.08 | 3.1 |
| Methoxsalen | 8.476 | 10.56 | 10.48 |
| Mitomycin | 1.122 | 1.003 | 0.912 |
| Mitotane | 5.932 | 11.01 | 10.17 |
| Mitoxantrone | 2.338 | 0.7284 | 0.6783 |
| Nelarabine | 8.659 | 11.04 | 10.08 |
| Nilotinib | 5.834 | 6.967 | 4.891 |
| Nutlin3 | 3.686 | 9.455 | 8.605 |
| Olaparib | 5.48 | 8.58 | 7.433 |
| Omacetaxine Mepesuccinate | 0.7749 | 0.7692 | 0.7717 |
| Osimertinib | 4.149 | 4.056 | 4.044 |
| Oxaliplatin | 2.372 | 5.272 | 5.118 |
| Paclitaxel | 1.453 | 1.453 | 1.432 |
| Palbociclib | 3.461 | 2.923 | 3.234 |
| Panobinostat | 0.6418 | 0.5862 | 0.5971 |
| Pazopanib Hydrochloride | 6.767 | 6.259 | 6.349 |
| Pemetrexed Disodium Salt | 2.501 | 2.438 | 2.852 |
| Heptahydrate | | | |
| Pentostatin | 10.31 | 9.855 | 9.32 |
| Pipobroman | 8.767 | 8.997 | 7.66 |
| Plerixafor | 9 | 8.893 | 8.524 |
| Plicamycin | 0.7344 | 0.767 | 0.6852 |
| Pomalidomide | 10.08 | 9.902 | 10.13 |
| Ponatinib | 3.312 | 2.944 | 3.632 |
| Pralatrexate | 2.822 | 2.774 | 2.86 |
| Procarbazine Hydrochloride | 8.119 | 10.04 | 10.7 |
| Raloxifene | 5.361 | 6.017 | 6.562 |
| Regorafenib | 3.995 | 5.541 | 5.25 |
| Romidepsin | 0.5862 | 0.6551 | 0.5264 |
| SenexinB | 5.203 | 6.869 | 6.954 |
| Sirolimus | 2.534 | 2.623 | 3.106 |
| Sorafenib | 3.981 | 5.27 | 4.98 |
| Streptozocin | 9.145 | 10.58 | 9.912 |
| Sunitinib | 3.556 | 3.076 | 3.758 |
| TamoxifenCitrate | 7.863 | 5.949 | 7.798 |
| Temozolomide | 9.187 | 9.565 | 10.06 |
| Temsirolimus | 2.486 | 1.939 | 2.767 |
| Teniposide | 1.342 | 0.9417 | 1.041 |
| Thalidomide | 7.13 | 9.18 | 10.21 |
| Thioguanine | 4.122 | 4.936 | 4.789 |
| Thiotepa | 3.965 | 5.443 | 5.535 |
| Topotecan Hydrochloride | 0.7919 | 0.7814 | 0.8044 |
| Trametinib | 7.726 | 6.641 | 8.533 |
| Tretinoin | 7.394 | 7.809 | 7.997 |
| Triethyleneme1amine | 2.223 | 2.224 | 2.319 |
| Trifluridine | 2.698 | 2.372 | 2.626 |
| UracilMustard | 9.331 | 9.337 | 9.516 |
| Valrubicin | 1.194 | 1.25 | 1.05 |
| Vandetanib | 2.871 | 2.831 | 3.022 |
| Vemurafenib | 8.722 | 7.987 | 8.365 |
| Vinblastine Sulfate | 1.393 | 1.54 | 1.382 |
| Vincristine Sulfate | 1.668 | 1.776 | 1.54 |
| Vinorelbine Tartrate | 1.616 | 1.797 | 1.629 |
| Vismodegib | 8.801 | 8.017 | 8.363 |
| Vorinostat | 2.073 | 2.202 | 2.114 |
| Zoledronic Acid | 9.222 | 9.268 | 9.417 |

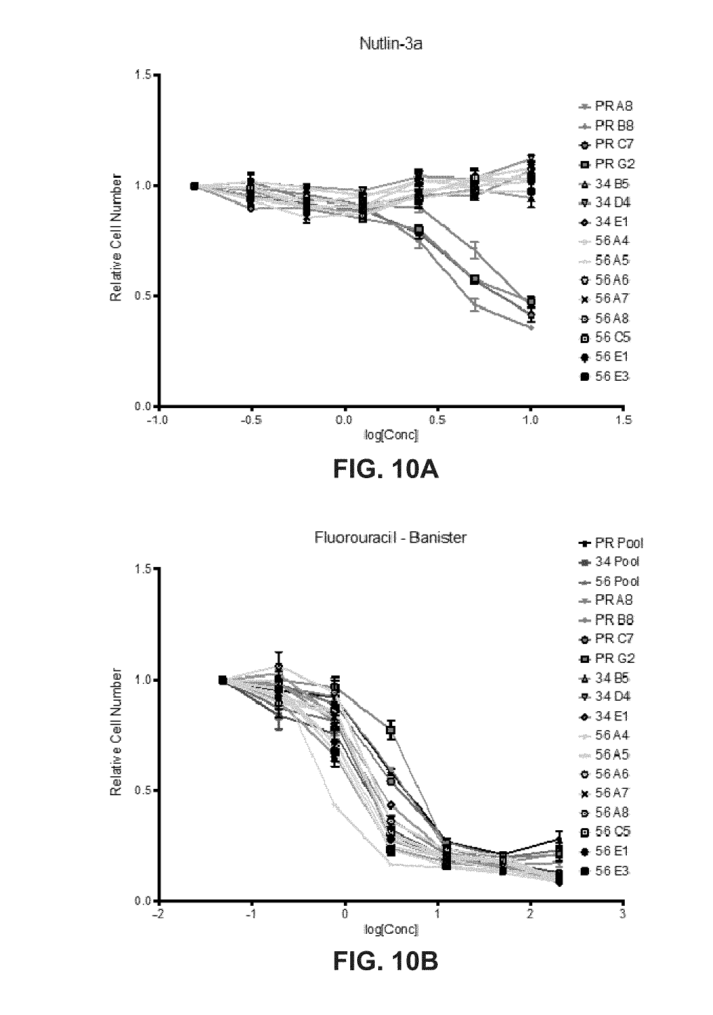

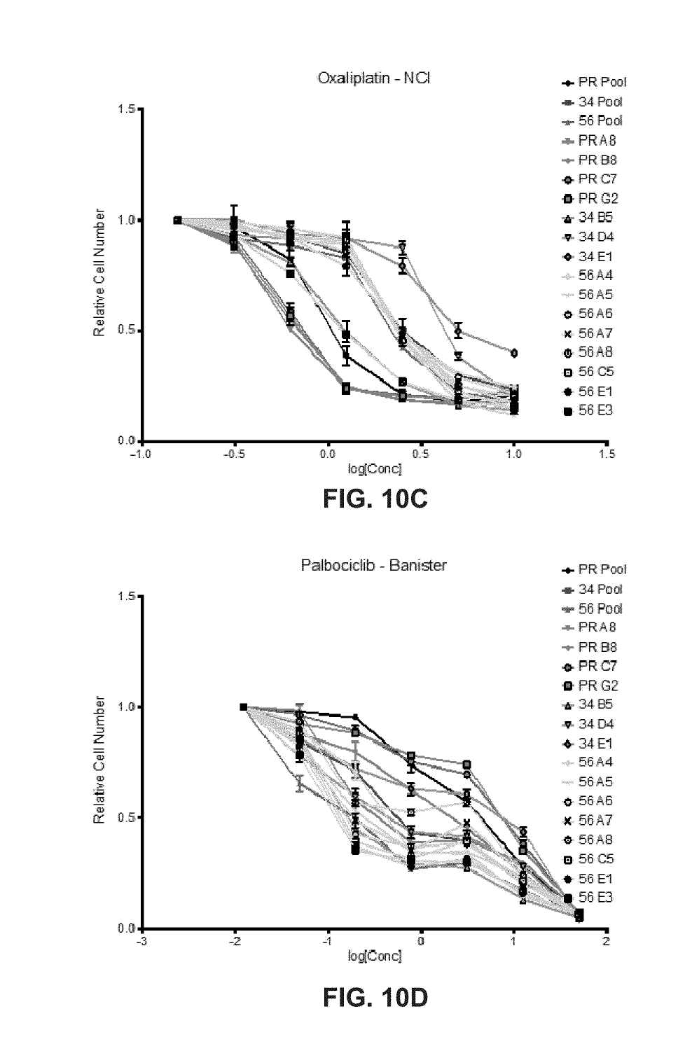

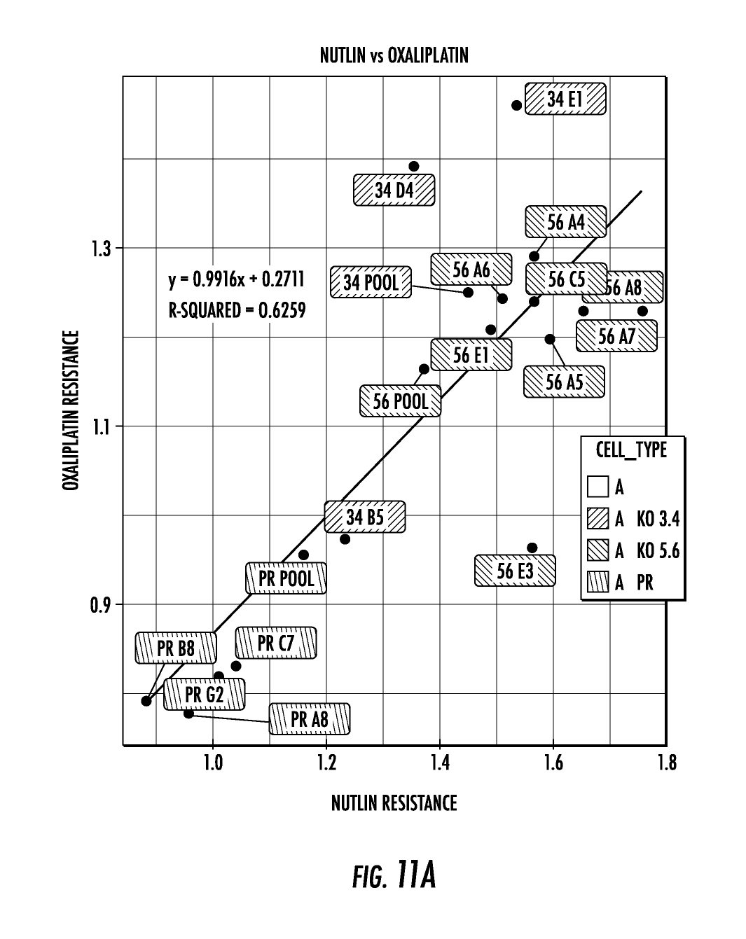

FIGS. 10A-10D illustrate sensitivities of MCF7 wild type and Knockout cell lines to Nutlin-3a, Fluorouracil, Oxaliplatin, and Palbociclib.

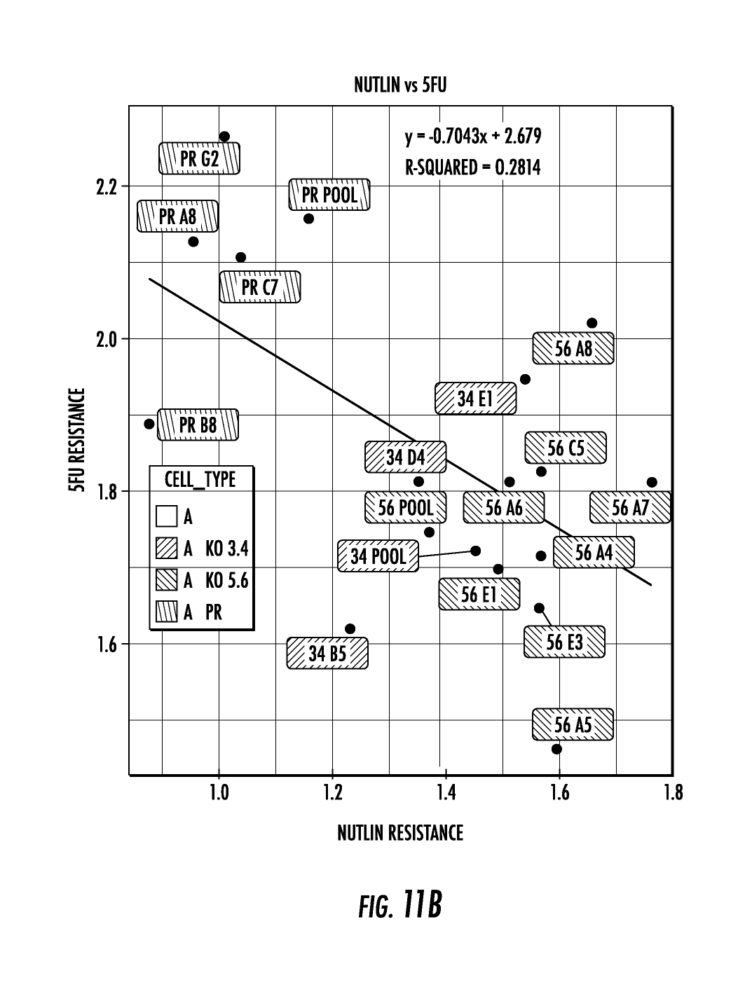

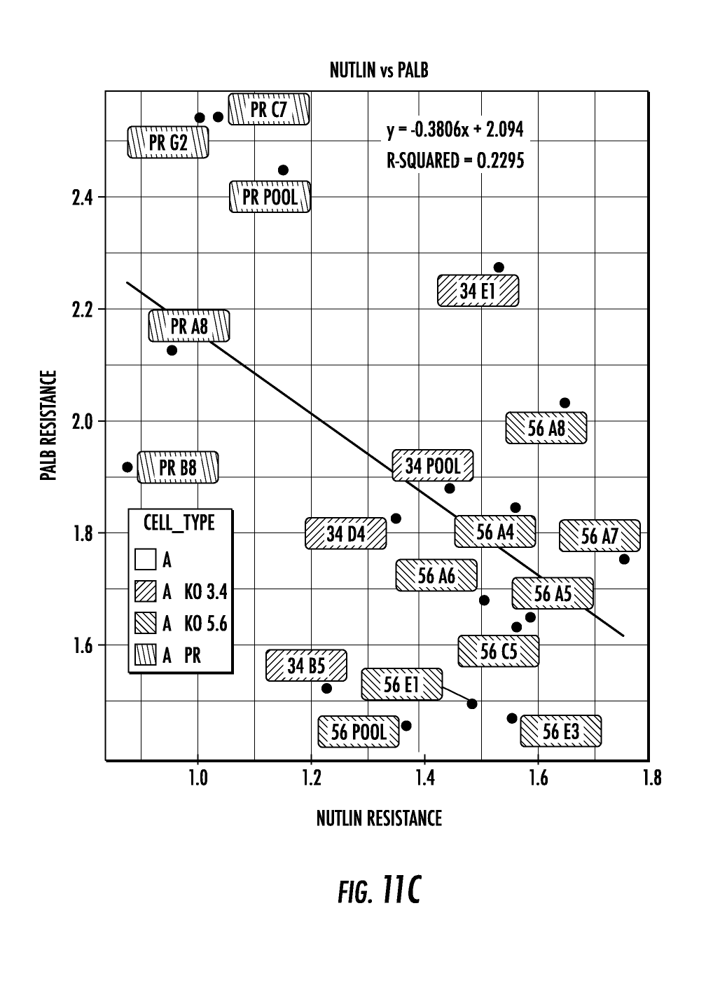

FIGS. 11A-11C illustrate that Nutlin resistance can be predictive of drug responses in oxaliplatin, fluorouracil (5FU), and palbociclib (Palb). Adjusted R-square values are shown. Values plotted are the areas under the curve for each treatment. See FIG. 8 and Table 3.

TABLE 3
Select anticancer drugs from the AOD-IV and their
respective areas under the curve (AUC) upon ten
day drug treatment on single cell clones.

| Cell line | 5-Fluorouracil | Nutlin-3a | Oxaliplatin | Palbociclib |
|---|---|---|---|---|
| PR WT Pool | 2.156 | 1.16 | 0.9557 | 2.446 |
| PR WT A8 | 2.126 | 0.9589 | 0.7779 | 2.127 |
| PR WT B8 | 1.886 | 0.8828 | 0.7911 | 1.917 |
| PR WT C7 | 2.105 | 1.042 | 0.8311 | 2.543 |
| PR WT G2 | 2.265 | 1.011 | 0.8186 | 2.542 |
| KO 34 Pool | 1.721 | 1.452 | 1.25 | 1.88 |
| KO 34 B5 | 1.618 | 1.234 | 0.973 | 1.522 |
| KO 34 D4 | 1.812 | 1.354 | 1.392 | 1.826 |
| KO 34 E1 | 1.945 | 1.538 | 1.46 | 2.274 |
| KO 56 Pool | 1.745 | 1.374 | 1.164 | 1.457 |
| KO 56 A4 | 1.716 | 1.568 | 1.29 | 1.846 |
| KO 56 A5 | 1.46 | 1.595 | 1.197 | 1.647 |
| KO 56 A6 | 1.811 | 1.512 | 1.243 | 1.681 |
| KO 56 A7 | 1.81 | 1.761 | 1.23 | 1.753 |
| KO 56 A8 | 2.02 | 1.656 | 1.229 | 2.031 |
| KO 56 C5 | 1.826 | 1.569 | 1.239 | 1.632 |
| KO 56 E1 | 1.697 | 1.492 | 1.208 | 1.497 |
| KO 56 E3 | 1.645 | 1.564 | 0.9635 | 1.469 |

While certain embodiments of the disclosed subject matter have been described using specific terms, such description is for illustrative purposes only, and it is to be understood that changes and variations may be made without departing from the spirit or scope of the subject matter.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only and is not intended to limit the invention further described in the appended claims.

1. A knockout cell line, wherein each cell of the knockout cell line comprises a MCF7 breast cancer cell having a deletion of at least one coding region in a gene, and wherein the gene includes a TP53 gene having a nucleotide sequence corresponding to Seq. ID No. 1.

2. The knockout cell line of claim 1, wherein the coding region comprises a part of an exon.

3. The knockout cell line of claim 1, wherein the coding region comprises one or more of exons 1-11.

4. The knockout cell line of claim 3, wherein each of exons on 1-11 include an nucleotide sequence from seq. ID No. 1, and wherein the nucleotide sequence corresponds to a range of base numbers for each exon selected from the group: the nucleotide sequence for exon 1 comprises base numbers 1-162, the nucleotide sequence for exon 2 comprises base numbers 10917-11018, the nucleotide sequence for exon 3 comprises base numbers 11136-11157, the nucleotide sequence for exon 4 comprises base numbers 11267-11545, the nucleotide sequence for exon 5 comprises base numbers 12303-12486, the nucleotide sequence for exon 6 comprises base numbers 12568-12680, the nucleotide sequence for exon 7 comprises base numbers 13249-13358, the nucleotide sequence for exon 8 comprises base numbers 13702-13838. the nucleotide sequence for exon 9 comprises base numbers 13931-14004, the nucleotide sequence for exon 10 comprises base numbers 16824-16930, the nucleotide sequence for exon 11 comprises base numbers 17849-19137.

5. The knockout cell line of claim 4, wherein the MCF7 breast cancer cell includes a deletion of the nucleotide sequence for exon 4, and wherein the nucleotide sequence for exon 4 comprises:
(SEQ ID NO: 5)
TCCC CCTTGCCGTC CCAAGCAATG
GATGATTTGA TGCTGTCCCC GGACGATATT GAACAATGGT
TCACTGAAGA CCCAGGTCCA GATGAAGCTC CCAGAATGCC
AGAGGCTGCT CCCCCCGTGG CCCCTGCACC AGCAGCTCCT
ACACCGGCGG CCCCTGCACC AGCCCCCTCC TGGCCCCTGT
CATCTTCTGT CCCTTCCCAG AAAACCTACC AGGGCAGCTA
CGGTTTCCGT CTGGGCTTCT TGCATTCTGG GACAGCCAAG
TCTGTGACTT GCACG

6. The knockout cell line of claim 4, wherein the MCF7 breast cancer cell includes a deletion of the nucleotide sequence for exon 10, and wherein the nucleotide sequence for exon 10 comprises:
(SEQ ID NO: 6)
ATCCGTG GGCGTGAGCG
CTTCGAGATG TTCCGAGAGC TGAATGAGGC CTTGGAACTC
AAGGATGCCC AGGCTGGGAA GGAGCCAGGG GGGAGCAGGG
CTCACTCCAG

7. An in vitro assay for determining efficacy of a treatment in breast cancer cells that include a TP53 gene mutation, comprising:
providing the treatment to a plurality of cells having a deletion of at least one coding region in the TP53 gene, and
measuring a result.

8. The in vitro assay of claim 7, further comprising:
providing the treatment to a plurality of wild type MCF7 breast cancer cells;
measuring a wild type result; and
comparing the wild type result to the result.

9. The in vitro assay of claim 8, wherein comparing the wild type result to the result comprises:
determining a first quantitative measurement describing the number of live wild type MCF7 breast cancer cells included in the plurality of wild type MCF7 breast cancer cells to which the treatment was provided;
determining a second quantitative measurement describing the number of live cells included in the plurality of cells having a deletion of at least one coding region in the TP53 gene to which the treatment was provided, and wherein the treatment provided to the wild type MCF7 breast cancer cells and the treatment provided to the plurality of cells having the deletion of at least one coding region in the TP53 gene are the same.

10. The in vitro assay of claim 7, wherein measuring the result comprises determining a quantitative measure of cell death.

11. The in vitro assay of claim 7, wherein providing the treatment comprises administering a drug to the plurality of cells derived from the knockout cell line.

12. The in vitro assay of claim 11, wherein the drug comprises one or more of the drugs from the group consisting of: Abiraterone, Afatanib, Alectinib, Allopurinol, Altretamine, Amifostine, Aminolevulinic Acid, Anastrozole, Arsenic Trioxide, Axitinib, Azactidine, Belinostat, Bendamustine Hydrochloride, Bleomycin Sulfate, Bortezomib, Bosutinib, Busulfan, Cabazitaxel, Cabozantinib, Capecitabine, Carboplatin, Carfilzomib, Carmustine, Celecoxib, Ceritinib, Chlorambucil, Cisplatin, Cladribine, Clofarabine, Clyclopamine, Cobimetinib, Crizotinib, Cyclophosphamide, Cytarabine Hydrochloride, Dabrafenib Mesylate, Dacarbazine, Dactinomycin, Dasatinib, Daunorubicin Hydrochloride, Decitabine, Dexrazoxane, DMSO, Docetaxel, Doxorubicin Hydrochloride, Enzalutamide, Epirubicin Hydrochloride, Erismodgib, Erlotinib Hydrochloride, Estramustine Phosphate Sodium, Etoposide, Everolimus, Exemestane, Floxuridine, Fludarabine Phosphate, Fluorouracil, Fulvestrant, Gefintinib, Gemcitabine Hydrochloride, Hydroxyurea, Ibrutinib, Idarubicin Hydrochloride, Idelalisib, Ifosfamide, Imatinib, Imiquimod, Irinotecan Hydrochloride, Ixabepilone, Ixazomib, Laptinib, Lenalidomide, Letrozole, Lomustine, Mechlorethamine Hydrochloride, Megestrol Acetate, Melphalan Hydrochloride, Mercaptopurine, Methotrexate, Methoxsalen, Mitomycin, Mitotane, Mitoxantrone, Nelarabine, Nilotinib, Nutlin3, Olaparib, Omacetaxine Mepesuccinate, Osimertinib, Oxaliplatin, Paclitaxel, Palbociclib, Panobinostat, Pazopanib Hydrochloride, Pemetrexed Disodium Salt Heptahydrate, Pentostatin, Pipobroman, Plerixafor, Plicamycin, Pomalidomide, Ponatinib, Pralatrexate, Procarbazine Hydrochloride, Raloxifene, Regorafenib, Rom idepsin, SenexinB, Sirolimus, Sorafenib, Streptozocin, Sunitinib, Tamoxifen Citrate, Temozolomide, Temsirolimus, Teniposide, Thalidomide, Thioguanine, Thiotepa, Topotecan Hydrochloride, Trametinib, Tretinoin, Triethylenemelamine, Trifluridine, Uracil Mustard, Valrubicin, Vandetanib, Vemurafenib, Vinblastine Sulfate, Vincristine Sulfate, Vinorelbine Tartrate, Vismodegib, Vorinostat, Zoledronic Acid 2.

13. A method for producing a knockout cell line from a wild type cell line, the method comprising deleting a portion of the TP53 gene in a cell derived from the wild type cell line, wherein deleting the portion of the TP53 gene comprises delivering a guide RNA to the cell.

14. The method of claim 13, wherein the wild type cell line comprises human MCF7 breast cancer cells.

15. The method of claim 13, wherein the portion of the TP53 gene comprises at least one coding region included in exons 4-10.

16. The method of claim 13, wherein delivering the guide RNA to the cell includes delivering an expression cassette to the cell, wherein the expression cassette includes a DNA sequence for expressing the guide RNA.

17. The method of claim 16, wherein delivering the guide RNA to the cell further includes delivering a second expression cassette to the cell, wherein the second expression cassette includes a DNA sequence for expressing Cas9.

18. The method of claim 13, further comprising selecting for a genetically modified cell, wherein selecting for the genetically modified cell comprises culturing the cell in the presence of an agent.

19. The method of claim 18, wherein the agent comprises Nutlin3.

20. The method of claim 13, wherein the guide RNA comprises one or more of the sequences:
(SEQ ID NO: 7)
CCATTGTTCAATATCGTCCG,
(SEQ ID NO: 8)
GACGGAAACCGTAGCTGCCC,
and
(SEQ ID NO: 9)
TGGTTATAGGATTCAACCGG.


SEQUENCE LISTING (37 sequences)

SEQ ID NO: 1 - DNA, 25760 bp, Homo sapiens
gttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccgcgggtcgcctgcaacctaggcggacgctaccatggcgtgagacagggagggaaagaagtgtgcagaaggcaagcccggaggtattttcaagaatgagtatatctcatcttcccggaggaaaaaaaaaaagaatgggtacgtctgagaatcaaattttgaaagagtgcaatgatgggtcgtttgataatttgtcggaaaaacaatctacctgttatctagctttgggctaggccattccagttccagacgcaggctgaacgtcgtgaagcggaaggggcgggcccgcaggcgtccgtgtggtcctccgtgcagccctccggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggctgggcgctgtggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgcgagtttggaagtaaaaagtagctgtattttgaactttttcagaagctctgagttttccagagcggttgtaccattttacactccaactagcaaggtatgggagttattatggttgtgccacagccttccggacattaggtattgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggttttttgtttgtttttattgttcagttgtgggactgctttatacattctggatacaagtcctttatcagatccatgtgtcgtgaatgttttcttctgatctgttgcttgcctatttgtttgctttacagagtttacagtatcttaagaggagtggatttatcttttttatgttcagtatttgccttgtcctgtttaggacatctttttttttttttttaaccccagggtcatgaagatattatcttacattttcttttaggacctttatggttgtaagttttacagtaaggtccttgagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaaatacaaaaagtagctaggcgtggtgacacatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccagcataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacaaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatattcaacacatatgcatgattacaaaccaaccaacctcctagcaaactagggaaaggaaacttaacctagtttgataacagggcgtccacagtcggagttccactagcagcatacataatggtagaaaactcagtgctgccgggcgcggtggctcacgcctgtaatgccagcactttgggaggcctaggcgggcggatcacgaggtcaggagatcgagactgtcctgactagcatgctgaaaccccgtctctactaaaaatacaaaaacaaaaaattagccgggcatggtggcgggcgcctatagtcccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagaccatcctggctaacacggtgaaaccctgtctctactaaatatacaaaaaattagccgggcgtagtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgcctataatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaatcccagcactttgggaggccgaggtgggtggatcacctgaggtcaggagttcaacaccagcctggccaacatggtgaaaccccatctctactaaaaatacaaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaacccaggaggcagaggttgcagtgagccgagattgtcccattgcactccagcctaggcgacaagagcaaaattccatgtcaaaaaaaaaaaaaaaaaaggaaagaaaaaaaataacgattagaaaggaagaaataaaacacattcacagccagtatgattctatacatacatgtcctaatggggccaggcgtggtggctcatgcctgtaatcctagcacttttaggaggctgaggcaggtggcttccctgggaccagcctggccaacatggtgaaaccccaactctaataaaaatacaaaaaatcagccaggcgtggtgacgggcacctctaatcccagctactcaggaggctgaggcaggagaattgcttggacctgggaggcagaggttgcagtgagccgagatcgcgctattgcactccagcctgggcaacaagagtgaaactccggcagggtgtggtggcttacgcctgtaatcccagcacttcgggaggctgaggcaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaaagagtgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggtatagtatttggatttaacctacacatatcctcccatatagtttaaattatctctagattacttacattacccccatacaatgaaaatgctaatgtacatgcaagtatgtatgtaagtacttgtactatattgtttagggaatcactggacatataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtacttgcattaatggagtgggggtgggagcagtaccacagatccgcactaacaatcacacagttctctctagaataataatatagaacaagtgaaatagaacaattgcagaaagagctaacctttgttgagctcttactgtgtgcccagcactttcctcaactctacatttcccataatacacagagtactaggtaggccaggcttggtggctcacgcctgtaatcccagcactttaggaggccaaggggggtggatcacctgaggtcgggagttcaagaccagcctgaccaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggcgcatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgaatctgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtccgaggctgtagtgagccatgattgcaccactgtaccctagcttgggcaacaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagccaggcatggtggcaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaagaaaacatggcaaagcctttgaaagcttgtctgggagaaggtgcgatgatagttgcataacttcgtgcaagatgctggtccacacaggggctgccccttgctctttctcgctctcttaacctctcatataacaggcttgtgtgttattcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaaagtctgcacgggaaggagcctacccccatgttcctggctagccaaggaaccaccagttgattagcagagaagggcagccagtctagctagagcttttggggaagagggagtggttgttaagagatgagattaaagaagccgagacgggccattcgtgaggggtttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggctgaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaaatacaggaagggagttgggaatagggtgcacatttaggaagtcttggggatttagtggtgggaaggttggaagtccctctctgattgtcttttcctcaaagaagtgcatggctggtgaggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaattgttaaagagagcatgaaaatggttctatgactttgcctgatacagatgctacttgacttacgatggtgttacttcctgataaactcgtcgtaagttgaaaatattgtaagttgaaaatggatttaatacacctaatctaaggaacatcatagcttagcctagcctgctttttttttttttttttttggagacagagtctcactctgtcacccaggctggagtgcagtggcgggatctcggctcactgcaacctccgccttctgggttcaagcgattctcctgcctcagcccactgagtagctgggattacaggcacctgccccgacgcccagctaattttttgttatttatttatttttttttttagtagagatgaggtttcaccatgttggccaggctagtctcgaactcctgaccttgtgatctgcctgccttggcctcccaaagtgctgggattacaggcgtgagccaccgcacccggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttatttttttgagactcagtctcactctgtcgcccaggctggagtgcagtggcatgatctcggctcactgcaacctctgcctcctgggttcaagtgattctcctgcctcagcctcccgaatagctaggactacaagcgcctgctaccacgcccagctaatttttgtatttttagtagagacagggtttcaccatgttggccaggctggtctcgaacttctgaccatgtgatccgcccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattgaatattgttactgaaagtgagaaacagcatggttgcatgaaaggaggcacagtcgagccaggcacagcctgggcgcagagcgagactcaaaaaaagaaaaggccaggcgcactggctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagatggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccaggtgtggtagcacacgcctgtagtcccagctactcgggaggctgaggcacaagaatcacttgaaccccagaggcggagattgcaatcagccaagattgcaccattgcactcccgcctgggcaacagagtgagaccccatctcaaaataaataaataaatatttttaaaagtcagctgtataggtacttgaagtgcagtttctactaaatgcatgttgcttttgtaccgtcataaagtcaaacaattgtaacttgaaccatcttttaactcaggtactgtgtatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttcctggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctcagtgtatccacagaacatccaaacccagggacgagtgtggatacttctttgccattctccgcaactcccagcccagagctggagggtctcaaggaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagataaataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtgaacgccagtgcaggctactggggtcagcaggtgcaggggtgagtgaggaggtgctgggaagcagccacctgagtctgcaatgagtgtgggctggggggcccagtgcccgggttccgggaggggaacaaaggctggagactgggtcagtctgcgggctgcatgacaacaagggagggggtggctccattcataactcaggaaccaaccgtccctcctcccctccggccacggctggcacaaggttctctccctcccctgcttctaggactgggctgcttccccctcggcagcctctcaccaaggattacgggatttaaatgtctgatttagcaaggctgagcctccagggtggccatctgctccatcagaaagtggcaggatacctgggttcccaaggggaacaggggtgggtgctactggatggagagaggccagtgggaggcctgctagccagggtcccaggaaagtgggggcagctaaggtaagagtaggggtgtggggctaggtccttcccagcatcccctcatcctgggcctcatgccaggtagctgaatgaattgaagctttaaactctgccaggaaaacctttcaaagggcttcttgggatagggaggagagtcgggttgaggagctcagtactgcctgcccatgctcctcagggctgctggctcccagggaggggggctgggagcaggcaggctcttccccatcacccactgctctcttggagccagtgcttgaaggggcagtcagacatggcttgcccttcctcctccctggtggtggagatgggtgttagggtccagtgggtgctactgtccaggggggcttctggggccaccagcctgtcagctcatcaaccaggctgaaggtgcaagcaggagccccttgccttgccccaaggatcccagacagctatgaagccaccagccttcctgacctcaagaccaccttttttttttctctttcttactagggaatgccaaacactctccccaggagatccagacccgcctctttcagagacttttaacttaaacatctgtccctacccagcaggcaaactagagctcctgaagctcagtccctgtccttgcctctgtagacaggtcaccttgatgagcttcctttttttttttttaatttttttttattttaggctttattggggcataattgatcccccaaaattgcatacattcaaggtatgcagtgtgatgatttgatatgggggtatattgtgaaaccattaccacaatcaaattaatcagcacgtccatcatcacacacagttaccatttgtgtgtgtgcacgtgtgttcacctacgacgaggacacttggacctactctgcagatctcaagtaaacagaaaatctccctttttgacaaccatcctccaccctttcaatcccaaccttttcctagattatgtccctagctctgtttttatttctgctgtgctgcttcagatccattctgactctgccaaacccttctttgtgagctgatagattgctggattgagaattacagctgggcgcggtggctcacgcctgtaatcccaacactgtgggaggccaaggccggcggatcacttgaggtcaggagttggagaccagcctgaccaacaagatgaaaccccatctctactaaaaatacaaaattagctgggcatggtggtgcacgcctgtaatctcatcttcttgggaggctgaggcaggagaattgcttgaacccgggaggtggaggttgcagtgagccaagatcctgccattgcactccagcctgggcaacaacagtgaagctccatctcaaaacacacaaaaaaaagaagtacaaagtctgagacttcaggccagctctgctacactatatactctaacctctctggtcctacttggtgacttctttccctctggtcgtgttcaagttcccgtcccatccagtcaagcaggtactcattggtaccttaccctgtgccaggagctgttctaggccctggaaacctatggcagacatgttccctaccctcccactcaaagagcccaggccttatcctaatgagatctgaaatcaaatctcccaatttcctcatggcttcagtctaaacttgtaattcacaaccttaaatcaatatgttctatttttttatttagaaaacatttccggccaggcacggtggatcacacctgtaatcccagctactcgggaggctgaggcaggagaatcgcttgaacccaggaggcagagggttgcagtgagccgagattgcgccattgcactctagcctgggcaacagagcaagactccatctcaaaaaagaaaaaaaaatggaagaaaaaaaaatttccccctcattttaggaacacgaggtctccaaatctaaaattcgtactctgaggagattgaatagccttaaatgctttcatcattaaaaagaaaagaaaggaacctggtatgcatcctaaaaatgaaaaatatacctacctgtaatcccagcacacagcacattgggaggctaaagcaggaggataacttgaggccaggagtttcagatcagcctgggcaacatagcaacaccccatttctttttcttttcttttttttttggagacacagtctcgctctgttactcaggctggagtgcagtggctcaatctcagctcactgcaagctctgcctcccaggttcatgccattctcctgcctcagcctcccgagtagctgggactacaggcgcccgccaccacgcctggctaattttttgtatttttagtagagacagggtttcaccgtgttagccaggatggtctcgatctcctgacctcgtgatccgccagccttggcctcctaaagtactgggattacaggcgtgagccactgcgcctggccacaacaccccatttctattttaataaaataaaatactgtgaaaaacatttacaatttttaaattttaattttaaaattaaacttatatttattcatttgtgtgtgtgggtttttttttttttttttttttgctttttttttgagatggagtgtcactctgtcacccaggctggagtgcagtggcgtgatctctgcctcccggttcaagtgattctcctgccatagcctcccaagtagctgggactacaggtacacgccaccacgccgggttaatttttgtatttttagtagagacaggatttcactgtgtcgccaggctagcctcgaactcctgacctcaggtgattcgcccaccttggcctcccaaagtgctgtgattacaagcgtgagccaccgtgcccagcccaaagttggttttaatagcagaaaatctatcaacataattcaatatattaaatttagaaagaaaaattatctatcatatcaacagatactgaaaggaatttgattaaatttcagtagccatttccttaaaaaagaaaacactttaacacagtaatagactgataatggaataccaattttcctaataagttaaacattaagataatttcaattaaggtcaagagctgggccaggtgcagtggctcacacctgtaatcccaacactttggaggccaaggtgggtggatcacctgaggtcaggagtggagaccagcctggctgacaatagtgaaatcctgcctctactaaaaacacaaaaaattagctgggcatggtggtgggcacctataatcccagctactgggaaggctgagacaggagaattgcttgaacctgggaggcggaggttgcagtgagcaaagatcacaccattgcactccagcctgggcgacagagccagagtcagtctcaaaaaaaaaaagaggtggccacacctataatccaaacattttgtgaggccaaggcaggagaattgcttcaggccaagagttgaacacctcgtcaacatagccagacctctctctagatagatagatagatgatagatagagagatagatagatgatagatagagagatagatagatgatagatagatagatagatagatagatagatagatagatagatagatagatagatagataatctggccgggtgtggaggctcacgcctgtaatcccagcactttgggaggctgaggcgggcagatcacgaggacaagagattgaaaccatcctggctaacaaggtgaaaccccgtctctactaaaaatacaaaaaattaggcgggtgtggtggcacgcgcctgtagtcctagctattcaggaggctgagacaggagaattgcttgaatccgaaaggcggaggttgcagcgagccgagatcgtgccactgcactccagcctgggtgacagagcaagactccatctcaaaataaataaataaataatcaagaacagtataaggggctgtatggtggctcatgcctgtgatcccagcactttgggaggccaaggtgggaggatcccttgagaccagcccaggcaacagagaaagaccctgtctctatttaaaaaaattaaaaactggccgggcacggtggctcacgcctgtaattccagcgcttgggaggccaaggcaggcacatcaggaggtcaggagttcgagaccagcctggccaacgtggtgaaaccccgtctctactaaaaatacaaaaagtagctaggcgtggtggcaggcacctgtaatcccagctacttgggaggctgaggcaggagaatcgcttgaacccaggaggcggaggttgcagtgggcaaagatcgtgccattgcactcagcctgggtgacagggcaagactccatctcaaaataaataaacaaagtaattaattaattaaattaaaaactgtggggatatagacttactctggttttattttttcttttcttttcttttcttttttctgagacggagtctcgctctgttgcccaggctggagtacagtggcgtggtttctgttctctgcaacctccacctcccggattcaagcgattctcttgcctcagcctcttgaatacctggaattacaggtgcctgccaccacccccggctaattttttgtatttttagtagagacagggtttcaccatgttggccaagctggtctcgaactcctgacctcatgatccacccgcctctgcctcccaaagcactgagactacaggagtgagccactgtgcccagcctactctggttttagtgcattcaagaggaacaaaaaaggaagaaaatcactagtaaatatacctctttctggttagagtggatgtttggaaattatatatatattatattatattatatatattatatatatacacaaacacgtacatacatgcacacacatatatgcctttttgattataggatagtataccaaaactcagaaatattatggaattaacagaatttagtaaggcagataagtagtaggtagaaaaatattaattttatcttccagcagaagcactgtgaaaaattagacaacaagaaaacattccattcaaaataatgacaataaggccgggcatggtggctcacacctgtaatcccagcactttgggaggctgaggcaggaggatcatctgaggtcaagtttgagatcagcctggccaacatggtgacaccctgtctctactgaaaatacaaaaatcagccagctatggtagtgtaagcctgtaattccagctactcgggaggtcgaagcagaagaatcacttgaacccaggaggcagagattgcagtgagccaagatcctgccagtgctttccagcctgggcaacagtgtgaggctccatctcaaaaaaaaaaaaaaaaaaaagacaatagcaataaacattaagaaatgtgtaataggaatggcacacacaaagaaggaatggcacagagcctgtatgcagaagaccacaaacccttatttaacgacgtaagccaagatccaaagaaaatgatagattctcagatgggaaaactaaaaaaataagaaaaatcaattatctcgagataaatataatataatgcaatttcaattagaatcccaaattttcattgtgtgtgtgtgtgagttgggtaaatttatcataaatgtataggaacgagtaagtgtcactagttgtttaaataaatactggatttgggccaggcatggtggctcacgcctctaatcccagcactttgggagaccgaggcgggcagatcatgaggtcaggagatcgagaccatctggccaacatagtgaaaactcgtctctactaaagatacaaaaaattagctgggcatggtggcacgtgcctgtagttccagctactctggaggctgaggcaggagagttgcttgaacccgggaggtggaggttgcaatgagccgagatcctgtcactgcactccaccctggcgacaaagtgagactccgtctctctctctctctttaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctc

SEQ ID NO: 2 - DNA, 25576 bp, Artificial Sequence
  Description of Artificial Sequence Synthetic polynucleotide
gttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccgcgggtcgcctgcaacctaggcggacgctaccatggcgtgagacagggagggaaagaagtgtgcagaaggcaagcccggaggtattttcaagaatgagtatatctcatcttcccggaggaaaaaaaaaaagaatgggtacgtctgagaatcaaattttgaaagagtgcaatgatgggtcgtttgataatttgtcggaaaaacaatctacctgttatctagctttgggctaggccattccagttccagacgcaggctgaacgtcgtgaagcggaaggggcgggcccgcaggcgtccgtgtggtcctccgtgcagccctccggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggctgggcgctgtggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgcgagtttggaagtaaaaagtagctgtattttgaactttttcagaagctctgagttttccagagcggttgtaccattttacactccaactagcaaggtatgggagttattatggttgtgccacagccttccggacattaggtattgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggttttttgtttgtttttattgttcagttgtgggactgctttatacattctggatacaagtcctttatcagatccatgtgtcgtgaatgttttcttctgatctgttgcttgcctatttgtttgctttacagagtttacagtatcttaagaggagtggatttatcttttttatgttcagtatttgccttgtcctgtttaggacatctttttttttttttttaaccccagggtcatgaagatattatcttacattttcttttaggacctttatggttgtaagttttacagtaaggtccttgagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaaatacaaaaagtagctaggcgtggtgacacatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccagcataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacaaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatattcaacacatatgcatgattacaaaccaaccaacctcctagcaaactagggaaaggaaacttaacctagtttgataacagggcgtccacagtcggagttccactagcagcatacataatggtagaaaactcagtgctgccgggcgcggtggctcacgcctgtaatgccagcactttgggaggcctaggcgggcggatcacgaggtcaggagatcgagactgtcctgactagcatgctgaaaccccgtctctactaaaaatacaaaaacaaaaaattagccgggcatggtggcgggcgcctatagtcccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagaccatcctggctaacacggtgaaaccctgtctctactaaatatacaaaaaattagccgggcgtagtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgcctataatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaatcccagcactttgggaggccgaggtgggtggatcacctgaggtcaggagttcaacaccagcctggccaacatggtgaaaccccatctctactaaaaatacaaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaacccaggaggcagaggttgcagtgagccgagattgtcccattgcactccagcctaggcgacaagagcaaaattccatgtcaaaaaaaaaaaaaaaaaaggaaagaaaaaaaataacgattagaaaggaagaaataaaacacattcacagccagtatgattctatacatacatgtcctaatggggccaggcgtggtggctcatgcctgtaatcctagcacttttaggaggctgaggcaggtggcttccctgggaccagcctggccaacatggtgaaaccccaactctaataaaaatacaaaaaatcagccaggcgtggtgacgggcacctctaatcccagctactcaggaggctgaggcaggagaattgcttggacctgggaggcagaggttgcagtgagccgagatcgcgctattgcactccagcctgggcaacaagagtgaaactccggcagggtgtggtggcttacgcctgtaatcccagcacttcgggaggctgaggcaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaaagagtgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggtatagtatttggatttaacctacacatatcctcccatatagtttaaattatctctagattacttacattacccccatacaatgaaaatgctaatgtacatgcaagtatgtatgtaagtacttgtactatattgtttagggaatcactggacatataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtacttgcattaatggagtgggggtgggagcagtaccacagatccgcactaacaatcacacagttctctctagaataataatatagaacaagtgaaatagaacaattgcagaaagagctaacctttgttgagctcttactgtgtgcccagcactttcctcaactctacatttcccataatacacagagtactaggtaggccaggcttggtggctcacgcctgtaatcccagcactttaggaggccaaggggggtggatcacctgaggtcgggagttcaagaccagcctgaccaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggcgcatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgaatctgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtccgaggctgtagtgagccatgattgcaccactgtaccctagcttgggcaacaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagccaggcatggtggcaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaagaaaacatggcaaagcctttgaaagcttgtctgggagaaggtgcgatgatagttgcataacttcgtgcaagatgctggtccacacaggggctgccccttgctctttctcgctctcttaacctctcatataacaggcttgtgtgttattcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaaagtctgcacgggaaggagcctacccccatgttcctggctagccaaggaaccaccagttgattagcagagaagggcagccagtctagctagagcttttggggaagagggagtggttgttaagagatgagattaaagaagccgagacgggccattcgtgaggggtttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggctgaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaaatacaggaagggagttgggaatagggtgcacatttaggaagtcttggggatttagtggtgggaaggttggaagtccctctctgattgtcttttcctcaaagaagtgcatggctggtgaggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccggacgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaattgttaaagagagcatgaaaatggttctatgactttgcctgatacagatgctacttgacttacgatggtgttacttcctgataaactcgtcgtaagttgaaaatattgtaagttgaaaatggatttaatacacctaatctaaggaacatcatagcttagcctagcctgctttttttttttttttttttggagacagagtctcactctgtcacccaggctggagtgcagtggcgggatctcggctcactgcaacctccgccttctgggttcaagcgattctcctgcctcagcccactgagtagctgggattacaggcacctgccccgacgcccagctaattttttgttatttatttatttttttttttagtagagatgaggtttcaccatgttggccaggctagtctcgaactcctgaccttgtgatctgcctgccttggcctcccaaagtgctgggattacaggcgtgagccaccgcacccggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttatttttttgagactcagtctcactctgtcgcccaggctggagtgcagtggcatgatctcggctcactgcaacctctgcctcctgggttcaagtgattctcctgcctcagcctcccgaatagctaggactacaagcgcctgctaccacgcccagctaatttttgtatttttagtagagacagggtttcaccatgttggccaggctggtctcgaacttctgaccatgtgatccgcccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattgaatattgttactgaaagtgagaaacagcatggttgcatgaaaggaggcacagtcgagccaggcacagcctgggcgcagagcgagactcaaaaaaagaaaaggccaggcgcactggctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagatggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccaggtgtggtagcacacgcctgtagtcccagctactcgggaggctgaggcacaagaatcacttgaaccccagaggcggagattgcaatcagccaagattgcaccattgcactcccgcctgggcaacagagtgagaccccatctcaaaataaataaataaatatttttaaaagtcagctgtataggtacttgaagtgcagtttctactaaatgcatgttgcttttgtaccgtcataaagtcaaacaattgtaacttgaaccatcttttaactcaggtactgtgtatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttcctggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctcagtgtatccacagaacatccaaacccagggacgagtgtggatacttctttgccattctccgcaactcccagcccagagctggagggtctcaaggaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagataaataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtgaacgccagtgcaggctactggggtcagcaggtgcaggggtgagtgaggaggtgctgggaagcagccacctgagtctgcaatgagtgtgggctggggggcccagtgcccgggttccgggaggggaacaaaggctggagactgggtcagtctgcgggctgcatgacaacaagggagggggtggctccattcataactcaggaaccaaccgtccctcctcccctccggccacggctggcacaaggttctctccctcccctgcttctaggactgggctgcttccccctcggcagcctctcaccaaggattacgggatttaaatgtctgatttagcaaggctgagcctccagggtggccatctgctccatcagaaagtggcaggatacctgggttcccaaggggaacaggggtgggtgctactggatggagagaggccagtgggaggcctgctagccagggtcccaggaaagtgggggcagctaaggtaagagtaggggtgtggggctaggtccttcccagcatcccctcatcctgggcctcatgccaggtagctgaatgaattgaagctttaaactctgccaggaaaacctttcaaagggcttcttgggatagggaggagagtcgggttgaggagctcagtactgcctgcccatgctcctcagggctgctggctcccagggaggggggctgggagcaggcaggctcttccccatcacccactgctctcttggagccagtgcttgaaggggcagtcagacatggcttgcccttcctcctccctggtggtggagatgggtgttagggtccagtgggtgctactgtccaggggggcttctggggccaccagcctgtcagctcatcaaccaggctgaaggtgcaagcaggagccccttgccttgccccaaggatcccagacagctatgaagccaccagccttcctgacctcaagaccaccttttttttttctctttcttactagggaatgccaaacactctccccaggagatccagacccgcctctttcagagacttttaacttaaacatctgtccctacccagcaggcaaactagagctcctgaagctcagtccctgtccttgcctctgtagacaggtcaccttgatgagcttcctttttttttttttaatttttttttattttaggctttattggggcataattgatcccccaaaattgcatacattcaaggtatgcagtgtgatgatttgatatgggggtatattgtgaaaccattaccacaatcaaattaatcagcacgtccatcatcacacacagttaccatttgtgtgtgtgcacgtgtgttcacctacgacgaggacacttggacctactctgcagatctcaagtaaacagaaaatctccctttttgacaaccatcctccaccctttcaatcccaaccttttcctagattatgtccctagctctgtttttatttctgctgtgctgcttcagatccattctgactctgccaaacccttctttgtgagctgatagattgctggattgagaattacagctgggcgcggtggctcacgcctgtaatcccaacactgtgggaggccaaggccggcggatcacttgaggtcaggagttggagaccagcctgaccaacaagatgaaaccccatctctactaaaaatacaaaattagctgggcatggtggtgcacgcctgtaatctcatcttcttgggaggctgaggcaggagaattgcttgaacccgggaggtggaggttgcagtgagccaagatcctgccattgcactccagcctgggcaacaacagtgaagctccatctcaaaacacacaaaaaaaagaagtacaaagtctgagacttcaggccagctctgctacactatatactctaacctctctggtcctacttggtgacttctttccctctggtcgtgttcaagttcccgtcccatccagtcaagcaggtactcattggtaccttaccctgtgccaggagctgttctaggccctggaaacctatggcagacatgttccctaccctcccactcaaagagcccaggccttatcctaatgagatctgaaatcaaatctcccaatttcctcatggcttcagtctaaacttgtaattcacaaccttaaatcaatatgttctatttttttatttagaaaacatttccggccaggcacggtggatcacacctgtaatcccagctactcgggaggctgaggcaggagaatcgcttgaacccaggaggcagagggttgcagtgagccgagattgcgccattgcactctagcctgggcaacagagcaagactccatctcaaaaaagaaaaaaaaatggaagaaaaaaaaatttccccctcattttaggaacacgaggtctccaaatctaaaattcgtactctgaggagattgaatagccttaaatgctttcatcattaaaaagaaaagaaaggaacctggtatgcatcctaaaaatgaaaaatatacctacctgtaatcccagcacacagcacattgggaggctaaagcaggaggataacttgaggccaggagtttcagatcagcctgggcaacatagcaacaccccatttctttttcttttcttttttttttggagacacagtctcgctctgttactcaggctggagtgcagtggctcaatctcagctcactgcaagctctgcctcccaggttcatgccattctcctgcctcagcctcccgagtagctgggactacaggcgcccgccaccacgcctggctaattttttgtatttttagtagagacagggtttcaccgtgttagccaggatggtctcgatctcctgacctcgtgatccgccagccttggcctcctaaagtactgggattacaggcgtgagccactgcgcctggccacaacaccccatttctattttaataaaataaaatactgtgaaaaacatttacaatttttaaattttaattttaaaattaaacttatatttattcatttgtgtgtgtgggtttttttttttttttttttttgctttttttttgagatggagtgtcactctgtcacccaggctggagtgcagtggcgtgatctctgcctcccggttcaagtgattctcctgccatagcctcccaagtagctgggactacaggtacacgccaccacgccgggttaatttttgtatttttagtagagacaggatttcactgtgtcgccaggctagcctcgaactcctgacctcaggtgattcgcccaccttggcctcccaaagtgctgtgattacaagcgtgagccaccgtgcccagcccaaagttggttttaatagcagaaaatctatcaacataattcaatatattaaatttagaaagaaaaattatctatcatatcaacagatactgaaaggaatttgattaaatttcagtagccatttccttaaaaaagaaaacactttaacacagtaatagactgataatggaataccaattttcctaataagttaaacattaagataatttcaattaaggtcaagagctgggccaggtgcagtggctcacacctgtaatcccaacactttggaggccaaggtgggtggatcacctgaggtcaggagtggagaccagcctggctgacaatagtgaaatcctgcctctactaaaaacacaaaaaattagctgggcatggtggtgggcacctataatcccagctactgggaaggctgagacaggagaattgcttgaacctgggaggcggaggttgcagtgagcaaagatcacaccattgcactccagcctgggcgacagagccagagtcagtctcaaaaaaaaaaagaggtggccacacctataatccaaacattttgtgaggccaaggcaggagaattgcttcaggccaagagttgaacacctcgtcaacatagccagacctctctctagatagatagatagatgatagatagagagatagatagatgatagatagagagatagatagatgatagatagatagatagatagatagatagatagatagatagatagatagatagatagataatctggccgggtgtggaggctcacgcctgtaatcccagcactttgggaggctgaggcgggcagatcacgaggacaagagattgaaaccatcctggctaacaaggtgaaaccccgtctctactaaaaatacaaaaaattaggcgggtgtggtggcacgcgcctgtagtcctagctattcaggaggctgagacaggagaattgcttgaatccgaaaggcggaggttgcagcgagccgagatcgtgccactgcactccagcctgggtgacagagcaagactccatctcaaaataaataaataaataatcaagaacagtataaggggctgtatggtggctcatgcctgtgatcccagcactttgggaggccaaggtgggaggatcccttgagaccagcccaggcaacagagaaagaccctgtctctatttaaaaaaattaaaaactggccgggcacggtggctcacgcctgtaattccagcgcttgggaggccaaggcaggcacatcaggaggtcaggagttcgagaccagcctggccaacgtggtgaaaccccgtctctactaaaaatacaaaaagtagctaggcgtggtggcaggcacctgtaatcccagctacttgggaggctgaggcaggagaatcgcttgaacccaggaggcggaggttgcagtgggcaaagatcgtgccattgcactcagcctgggtgacagggcaagactccatctcaaaataaataaacaaagtaattaattaattaaattaaaaactgtggggatatagacttactctggttttattttttcttttcttttcttttcttttttctgagacggagtctcgctctgttgcccaggctggagtacagtggcgtggtttctgttctctgcaacctccacctcccggattcaagcgattctcttgcctcagcctcttgaatacctggaattacaggtgcctgccaccacccccggctaattttttgtatttttagtagagacagggtttcaccatgttggccaagctggtctcgaactcctgacctcatgatccacccgcctctgcctcccaaagcactgagactacaggagtgagccactgtgcccagcctactctggttttagtgcattcaagaggaacaaaaaaggaagaaaatcactagtaaatatacctctttctggttagagtggatgtttggaaattatatatatattatattatattatatatattatatatatacacaaacacgtacatacatgcacacacatatatgcctttttgattataggatagtataccaaaactcagaaatattatggaattaacagaatttagtaaggcagataagtagtaggtagaaaaatattaattttatcttccagcagaagcactgtgaaaaattagacaacaagaaaacattccattcaaaataatgacaataaggccgggcatggtggctcacacctgtaatcccagcactttgggaggctgaggcaggaggatcatctgaggtcaagtttgagatcagcctggccaacatggtgacaccctgtctctactgaaaatacaaaaatcagccagctatggtagtgtaagcctgtaattccagctactcgggaggtcgaagcagaagaatcacttgaacccaggaggcagagattgcagtgagccaagatcctgccagtgctttccagcctgggcaacagtgtgaggctccatctcaaaaaaaaaaaaaaaaaaaagacaatagcaataaacattaagaaatgtgtaataggaatggcacacacaaagaaggaatggcacagagcctgtatgcagaagaccacaaacccttatttaacgacgtaagccaagatccaaagaaaatgatagattctcagatgggaaaactaaaaaaataagaaaaatcaattatctcgagataaatataatataatgcaatttcaattagaatcccaaattttcattgtgtgtgtgtgtgagttgggtaaatttatcataaatgtataggaacgagtaagtgtcactagttgtttaaataaatactggatttgggccaggcatggtggctcacgcctctaatcccagcactttgggagaccgaggcgggcagatcatgaggtcaggagatcgagaccatctggccaacatagtgaaaactcgtctctactaaagatacaaaaaattagctgggcatggtggcacgtgcctgtagttccagctactctggaggctgaggcaggagagttgcttgaacccgggaggtggaggttgcaatgagccgagatcctgtcactgcactccaccctggcgacaaagtgagactccgtctctctctctctctttaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctc

SEQ ID NO: 3 - DNA, 19984 bp, Artificial Sequence
  Description of Artificial Sequence Synthetic polynucleotide
gttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccgcgggtcgcctgcaacctaggcggacgctaccatggcgtgagacagggagggaaagaagtgtgcagaaggcaagcccggaggtattttcaagaatgagtatatctcatcttcccggaggaaaaaaaaaaagaatgggtacgtctgagaatcaaattttgaaagagtgcaatgatgggtcgtttgataatttgtcggaaaaacaatctacctgttatctagctttgggctaggccattccagttccagacgcaggctgaacgtcgtgaagcggaaggggcgggcccgcaggcgtccgtgtggtcctccgtgcagccctccggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggctgggcgctgtggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgcgagtttggaagtaaaaagtagctgtattttgaactttttcagaagctctgagttttccagagcggttgtaccattttacactccaactagcaaggtatgggagttattatggttgtgccacagccttccggacattaggtattgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggttttttgtttgtttttattgttcagttgtgggactgctttatacattctggatacaagtcctttatcagatccatgtgtcgtgaatgttttcttctgatctgttgcttgcctatttgtttgctttacagagtttacagtatcttaagaggagtggatttatcttttttatgttcagtatttgccttgtcctgtttaggacatctttttttttttttttaaccccagggtcatgaagatattatcttacattttcttttaggacctttatggttgtaagttttacagtaaggtccttgagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaaatacaaaaagtagctaggcgtggtgacacatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccagcataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacaaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatattcaacacatatgcatgattacaaaccaaccaacctcctagcaaactagggaaaggaaacttaacctagtttgataacagggcgtccacagtcggagttccactagcagcatacataatggtagaaaactcagtgctgccgggcgcggtggctcacgcctgtaatgccagcactttgggaggcctaggcgggcggatcacgaggtcaggagatcgagactgtcctgactagcatgctgaaaccccgtctctactaaaaatacaaaaacaaaaaattagccgggcatggtggcgggcgcctatagtcccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagaccatcctggctaacacggtgaaaccctgtctctactaaatatacaaaaaattagccgggcgtagtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgcctataatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaatcccagcactttgggaggccgaggtgggtggatcacctgaggtcaggagttcaacaccagcctggccaacatggtgaaaccccatctctactaaaaatacaaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaacccaggaggcagaggttgcagtgagccgagattgtcccattgcactccagcctaggcgacaagagcaaaattccatgtcaaaaaaaaaaaaaaaaaaggaaagaaaaaaaataacgattagaaaggaagaaataaaacacattcacagccagtatgattctatacatacatgtcctaatggggccaggcgtggtggctcatgcctgtaatcctagcacttttaggaggctgaggcaggtggcttccctgggaccagcctggccaacatggtgaaaccccaactctaataaaaatacaaaaaatcagccaggcgtggtgacgggcacctctaatcccagctactcaggaggctgaggcaggagaattgcttggacctgggaggcagaggttgcagtgagccgagatcgcgctattgcactccagcctgggcaacaagagtgaaactccggcagggtgtggtggcttacgcctgtaatcccagcacttcgggaggctgaggcaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaaagagtgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggtatagtatttggatttaacctacacatatcctcccatatagtttaaattatctctagattacttacattacccccatacaatgaaaatgctaatgtacatgcaagtatgtatgtaagtacttgtactatattgtttagggaatcactggacatataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtacttgcattaatggagtgggggtgggagcagtaccacagatccgcactaacaatcacacagttctctctagaataataatatagaacaagtgaaatagaacaattgcagaaagagctaacctttgttgagctcttactgtgtgcccagcactttcctcaactctacatttcccataatacacagagtactaggtaggccaggcttggtggctcacgcctgtaatcccagcactttaggaggccaaggggggtggatcacctgaggtcgggagttcaagaccagcctgaccaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggcgcatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgaatctgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtccgaggctgtagtgagccatgattgcaccactgtaccctagcttgggcaacaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagccaggcatggtggcaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaagaaaacatggcaaagcctttgaaagcttgtctgggagaaggtgcgatgatagttgcataacttcgtgcaagatgctggtccacacaggggctgccccttgctctttctcgctctcttaacctctcatataacaggcttgtgtgttattcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaaagtctgcacgggaaggagcctacccccatgttcctggctagccaaggaaccaccagttgattagcagagaagggcagccagtctagctagagcttttggggaagagggagtggttgttaagagatgagattaaagaagccgagacgggccattcgtgaggggtttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggctgaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaaatacaggaagggagttgggaatagggtgcacatttaggaagtcttggggatttagtggtgggaaggttggaagtccctctctgattgtcttttcctcaaagaagtgcatggctggtgaggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccgttgaatcctataaccacattcttgcctcagtgtatccacagaacatccaaacccagggacgagtgtggatacttctttgccattctccgcaactcccagcccagagctggagggtctcaaggaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagataaataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtgaacgccagtgcaggctactggggtcagcaggtgcaggggtgagtgaggaggtgctgggaagcagccacctgagtctgcaatgagtgtgggctggggggcccagtgcccgggttccgggaggggaacaaaggctggagactgggtcagtctgcgggctgcatgacaacaagggagggggtggctccattcataactcaggaaccaaccgtccctcctcccctccggccacggctggcacaaggttctctccctcccctgcttctaggactgggctgcttccccctcggcagcctctcaccaaggattacgggatttaaatgtctgatttagcaaggctgagcctccagggtggccatctgctccatcagaaagtggcaggatacctgggttcccaaggggaacaggggtgggtgctactggatggagagaggccagtgggaggcctgctagccagggtcccaggaaagtgggggcagctaaggtaagagtaggggtgtggggctaggtccttcccagcatcccctcatcctgggcctcatgccaggtagctgaatgaattgaagctttaaactctgccaggaaaacctttcaaagggcttcttgggatagggaggagagtcgggttgaggagctcagtactgcctgcccatgctcctcagggctgctggctcccagggaggggggctgggagcaggcaggctcttccccatcacccactgctctcttggagccagtgcttgaaggggcagtcagacatggcttgcccttcctcctccctggtggtggagatgggtgttagggtccagtgggtgctactgtccaggggggcttctggggccaccagcctgtcagctcatcaaccaggctgaaggtgcaagcaggagccccttgccttgccccaaggatcccagacagctatgaagccaccagccttcctgacctcaagaccaccttttttttttctctttcttactagggaatgccaaacactctccccaggagatccagacccgcctctttcagagacttttaacttaaacatctgtccctacccagcaggcaaactagagctcctgaagctcagtccctgtccttgcctctgtagacaggtcaccttgatgagcttcctttttttttttttaatttttttttattttaggctttattggggcataattgatcccccaaaattgcatacattcaaggtatgcagtgtgatgatttgatatgggggtatattgtgaaaccattaccacaatcaaattaatcagcacgtccatcatcacacacagttaccatttgtgtgtgtgcacgtgtgttcacctacgacgaggacacttggacctactctgcagatctcaagtaaacagaaaatctccctttttgacaaccatcctccaccctttcaatcccaaccttttcctagattatgtccctagctctgtttttatttctgctgtgctgcttcagatccattctgactctgccaaacccttctttgtgagctgatagattgctggattgagaattacagctgggcgcggtggctcacgcctgtaatcccaacactgtgggaggccaaggccggcggatcacttgaggtcaggagttggagaccagcctgaccaacaagatgaaaccccatctctactaaaaatacaaaattagctgggcatggtggtgcacgcctgtaatctcatcttcttgggaggctgaggcaggagaattgcttgaacccgggaggtggaggttgcagtgagccaagatcctgccattgcactccagcctgggcaacaacagtgaagctccatctcaaaacacacaaaaaaaagaagtacaaagtctgagacttcaggccagctctgctacactatatactctaacctctctggtcctacttggtgacttctttccctctggtcgtgttcaagttcccgtcccatccagtcaagcaggtactcattggtaccttaccctgtgccaggagctgttctaggccctggaaacctatggcagacatgttccctaccctcccactcaaagagcccaggccttatcctaatgagatctgaaatcaaatctcccaatttcctcatggcttcagtctaaacttgtaattcacaaccttaaatcaatatgttctatttttttatttagaaaacatttccggccaggcacggtggatcacacctgtaatcccagctactcgggaggctgaggcaggagaatcgcttgaacccaggaggcagagggttgcagtgagccgagattgcgccattgcactctagcctgggcaacagagcaagactccatctcaaaaaagaaaaaaaaatggaagaaaaaaaaatttccccctcattttaggaacacgaggtctccaaatctaaaattcgtactctgaggagattgaatagccttaaatgctttcatcattaaaaagaaaagaaaggaacctggtatgcatcctaaaaatgaaaaatatacctacctgtaatcccagcacacagcacattgggaggctaaagcaggaggataacttgaggccaggagtttcagatcagcctgggcaacatagcaacaccccatttctttttcttttcttttttttttggagacacagtctcgctctgttactcaggctggagtgcagtggctcaatctcagctcactgcaagctctgcctcccaggttcatgccattctcctgcctcagcctcccgagtagctgggactacaggcgcccgccaccacgcctggctaattttttgtatttttagtagagacagggtttcaccgtgttagccaggatggtctcgatctcctgacctcgtgatccgccagccttggcctcctaaagtactgggattacaggcgtgagccactgcgcctggccacaacaccccatttctattttaataaaataaaatactgtgaaaaacatttacaatttttaaattttaattttaaaattaaacttatatttattcatttgtgtgtgtgggtttttttttttttttttttttgctttttttttgagatggagtgtcactctgtcacccaggctggagtgcagtggcgtgatctctgcctcccggttcaagtgattctcctgccatagcctcccaagtagctgggactacaggtacacgccaccacgccgggttaatttttgtatttttagtagagacaggatttcactgtgtcgccaggctagcctcgaactcctgacctcaggtgattcgcccaccttggcctcccaaagtgctgtgattacaagcgtgagccaccgtgcccagcccaaagttggttttaatagcagaaaatctatcaacataattcaatatattaaatttagaaagaaaaattatctatcatatcaacagatactgaaaggaatttgattaaatttcagtagccatttccttaaaaaagaaaacactttaacacagtaatagactgataatggaataccaattttcctaataagttaaacattaagataatttcaattaaggtcaagagctgggccaggtgcagtggctcacacctgtaatcccaacactttggaggccaaggtgggtggatcacctgaggtcaggagtggagaccagcctggctgacaatagtgaaatcctgcctctactaaaaacacaaaaaattagctgggcatggtggtgggcacctataatcccagctactgggaaggctgagacaggagaattgcttgaacctgggaggcggaggttgcagtgagcaaagatcacaccattgcactccagcctgggcgacagagccagagtcagtctcaaaaaaaaaaagaggtggccacacctataatccaaacattttgtgaggccaaggcaggagaattgcttcaggccaagagttgaacacctcgtcaacatagccagacctctctctagatagatagatagatgatagatagagagatagatagatgatagatagagagatagatagatgatagatagatagatagatagatagatagatagatagatagatagatagatagatagataatctggccgggtgtggaggctcacgcctgtaatcccagcactttgggaggctgaggcgggcagatcacgaggacaagagattgaaaccatcctggctaacaaggtgaaaccccgtctctactaaaaatacaaaaaattaggcgggtgtggtggcacgcgcctgtagtcctagctattcaggaggctgagacaggagaattgcttgaatccgaaaggcggaggttgcagcgagccgagatcgtgccactgcactccagcctgggtgacagagcaagactccatctcaaaataaataaataaataatcaagaacagtataaggggctgtatggtggctcatgcctgtgatcccagcactttgggaggccaaggtgggaggatcccttgagaccagcccaggcaacagagaaagaccctgtctctatttaaaaaaattaaaaactggccgggcacggtggctcacgcctgtaattccagcgcttgggaggccaaggcaggcacatcaggaggtcaggagttcgagaccagcctggccaacgtggtgaaaccccgtctctactaaaaatacaaaaagtagctaggcgtggtggcaggcacctgtaatcccagctacttgggaggctgaggcaggagaatcgcttgaacccaggaggcggaggttgcagtgggcaaagatcgtgccattgcactcagcctgggtgacagggcaagactccatctcaaaataaataaacaaagtaattaattaattaaattaaaaactgtggggatatagacttactctggttttattttttcttttcttttcttttcttttttctgagacggagtctcgctctgttgcccaggctggagtacagtggcgtggtttctgttctctgcaacctccacctcccggattcaagcgattctcttgcctcagcctcttgaatacctggaattacaggtgcctgccaccacccccggctaattttttgtatttttagtagagacagggtttcaccatgttggccaagctggtctcgaactcctgacctcatgatccacccgcctctgcctcccaaagcactgagactacaggagtgagccactgtgcccagcctactctggttttagtgcattcaagaggaacaaaaaaggaagaaaatcactagtaaatatacctctttctggttagagtggatgtttggaaattatatatatattatattatattatatatattatatatatacacaaacacgtacatacatgcacacacatatatgcctttttgattataggatagtataccaaaactcagaaatattatggaattaacagaatttagtaaggcagataagtagtaggtagaaaaatattaattttatcttccagcagaagcactgtgaaaaattagacaacaagaaaacattccattcaaaataatgacaataaggccgggcatggtggctcacacctgtaatcccagcactttgggaggctgaggcaggaggatcatctgaggtcaagtttgagatcagcctggccaacatggtgacaccctgtctctactgaaaatacaaaaatcagccagctatggtagtgtaagcctgtaattccagctactcgggaggtcgaagcagaagaatcacttgaacccaggaggcagagattgcagtgagccaagatcctgccagtgctttccagcctgggcaacagtgtgaggctccatctcaaaaaaaaaaaaaaaaaaaagacaatagcaataaacattaagaaatgtgtaataggaatggcacacacaaagaaggaatggcacagagcctgtatgcagaagaccacaaacccttatttaacgacgtaagccaagatccaaagaaaatgatagattctcagatgggaaaactaaaaaaataagaaaaatcaattatctcgagataaatataatataatgcaatttcaattagaatcccaaattttcattgtgtgtgtgtgtgagttgggtaaatttatcataaatgtataggaacgagtaagtgtcactagttgtttaaataaatactggatttgggccaggcatggtggctcacgcctctaatcccagcactttgggagaccgaggcgggcagatcatgaggtcaggagatcgagaccatctggccaacatagtgaaaactcgtctctactaaagatacaaaaaattagctgggcatggtggcacgtgcctgtagttccagctactctggaggctgaggcaggagagttgcttgaacccgggaggtggaggttgcaatgagccgagatcctgtcactgcactccaccctggcgacaaagtgagactccgtctctctctctctctttaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctc

SEQ ID NO: 4 - DNA, 188 bp, Homo sapiens
ggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttcc

SEQ ID NO: 5 - DNA, 279 bp, Homo sapiens
tcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacg

SEQ ID NO: 6 - DNA, 107 bp, Homo sapiens
atccgtgggcgtgagcgcttcgagatgttccgagagctgaatgaggccttggaactcaaggatgcccaggctgggaaggagccaggggggagcagggctcactccag

SEQ ID NO: 7 - DNA, 20 bp, Artificial Sequence
  Description of Artificial Sequence Synthetic oligonucleotide
ccattgttcaatatcgtccg

SEQ ID NO: 8 - DNA, 20 bp, Artificial Sequence
  Description of Artificial Sequence Synthetic oligonucleotide
gacggaaaccgtagctgccc

SEQ ID NO: 9 - DNA, 20 bp, Artificial Sequence
  Description of Artificial Sequence Synthetic oligonucleotide
tggttataggattcaaccgg

SEQ ID NO: 10 - DNA, 19 bp, Artificial Sequence
  Description of Artificial Sequence Synthetic oligonucleotide
cattgttcaatatcgtccg

SEQ ID NO: 11 - DNA, 162 bp, Homo sapiens
gttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgg

SEQ ID NO: 12 - DNA, 102 bp, Homo sapiens
cagccagactgccttccgggtcactgccatggaggagccgcagtcagatcctagcgtcgagccccctctgagtcaggaaacattttcagacctatggaaact

SEQ ID NO: 13 - DNA, 22 bp, Homo sapiens
acttcctgaaaacaacgttctg

SEQ ID NO: 14 - DNA, 184 bp, Homo sapiens
tactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatg

SEQ ID NO: 15 - DNA, 113 bp, Homo sapiens
gtctggcccctcctcagcatcttatccgagtggaaggaaatttgcgtgtggagtatttggatgacagaaacacttttcgacatagtgtggtggtgccctatgagccgcctgag

SEQ ID NO: 16 - DNA, 110 bp, Homo sapiens
gttggctctgactgtaccaccatccactacaactacatgtgtaacagttcctgcatgggcggcatgaaccggaggcccatcctcaccatcatcacactggaagactccag

SEQ ID NO: 17 - DNA, 137 bp, Homo sapiens
tggtaatctactgggacggaacagctttgaggtgcgtgtttgtgcctgtcctgggagagaccggcgcacagaggaagagaatctccgcaagaaaggggagcctcaccacgagctgcccccagggagcactaagcgag

SEQ ID NO: 18 - DNA, 74 bp, Homo sapiens
cactgcccaacaacaccagctcctctccccagccaaagaagaaaccactggatggagaatatttcacccttcag

SEQ ID NO: 19 - DNA, 1289 bp, Homo sapiens
ccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 20 - DNA, 24 bp, Artificial Sequence
  Description of Artificial Sequence Synthetic primer
gtacaaaaaagcaggctttaaagg

SEQ ID NO: 21 - DNA, 25 bp, Artificial Sequence
  Description of Artificial Sequence Synthetic primer
taatgccaactttgtacaagaaagc

SEQ ID NO: 22 - DNA, 20 bp, Artificial Sequence
  Description of Artificial Sequence Synthetic primer
ctggtaaggacaagggttgg

SEQ ID NO: 23 - DNA, 20 bp, Artificial Sequence
  Description of Artificial Sequence Synthetic primer
gccaaagggtgaagaggaat

SEQ ID NO: 24 - DNA, 20 bp, Artificial Sequence
  Description of Artificial Sequence Synthetic primer
attaggcccctccttgagac

SEQ ID NO: 25 - DNA, 34 bp, Artificial Sequence
  Description of Artificial Sequence Synthetic oligonucleotide
ctgtccccggacgatattgaacaatggttcactg

SEQ ID NO: 26 - DNA, 14 bp, Artificial Sequence
  Description of Artificial Sequence Synthetic oligonucleotide
tcgtccggggacag

SEQ ID NO: 27 - DNA, 37 bp, Artificial Sequence
  Description of Artificial Sequence Synthetic oligonucleotide
tagtcttcctccggttgaatcctataaccacattctt

SEQ ID NO: 28 - DNA, 30 bp, Artificial Sequence
  Description of Artificial Sequence Synthetic oligonucleotide
aagaatgtggttataggattcaaccggagg

SEQ ID NO: 29 - DNA, 49 bp, Artificial Sequence
  Description of Artificial Sequence Synthetic oligonucleotide
ttgatgctgtccccgggttgaatcctataaccacattcttgcctcagtg

SEQ ID NO: 30 - DNA, 32 bp, Artificial Sequence
  Description of Artificial Sequence Synthetic oligonucleotide
gtccccggacgatattgaacaatggttcactg

SEQ ID NO: 31 - DNA, 14 bp, Artificial Sequence
  Description of Artificial Sequence Synthetic oligonucleotide
tatcgtccggggac

SEQ ID NO: 32 - DNA, 28 bp, Artificial Sequence
  Description of Artificial Sequence Synthetic oligonucleotide
accagggcagctacggtttccgtctggg

SEQ ID NO: 33 - DNA, 41 bp, Artificial Sequence
  Description of Artificial Sequence Synthetic oligonucleotide
gatttgatgctgtccccggacgtctgggcttcttgctttct

SEQ ID NO: 34 - DNA, 41 bp, Artificial Sequence
  Description of Artificial Sequence Synthetic oligonucleotide
gatttgatgctgttcccggacgtctgggcttcttgcattct

SEQ ID NO: 35 - DNA, 41 bp, Artificial Sequence
  Description of Artificial Sequence Synthetic oligonucleotide
gatttgatgctgtccccggacgtctgggcttcttgcattct

SEQ ID NO: 36 - DNA, 36 bp, Artificial Sequence
  Description of Artificial Sequence Synthetic oligonucleotide
tgtccccggaacgatattgaacaatggttcactgaa

SEQ ID NO: 37 - DNA, 35 bp, Artificial Sequence
  Description of Artificial Sequence Synthetic oligonucleotide
tgtccccggacgatattgaacaatggttcactgaa